(19) United States
(12) Patent Application Publication
(10) Pub. No.: US 2009/0149526 A1
(43) Pub. Date: Jun. 11, 2009

(54) Tetracyclic Indole Derivatives as Antiviral Agents

(76) Inventors: Frank Narjes, Pomezia (Rome) (IT); Ian Stansfield, Pomezia (Rome) (IT)

Correspondence Address: MERCK AND CO., INC, P O BOX 2000, RAHWAY, NJ, 07065-0907, US

(21) Appl. No.: 11/991,526

(22) PCT Filed: Sep. 1, 2006
(86) PCT No.: PCT/GB2006/050267

§ 371 Date: Mar. 6, 2008

(30) Foreign Application Priority Data
Sep. 9, 2005 (GB) 0518390.9

(51) Int. Cl. A61K 31/407; C07D 487/04; A61P 31/12; C07D 498/04

(52) U.S. Cl. 514/410; 540/471; 540/468

(57) ABSTRACT

The present invention relates to tetracyclic indole derivatives of formula (I): wherein Ar, A, R¹, R², L, W, X, Y and Z are defined herein, and pharmaceutically acceptable salts thereof, pharmaceutical compositions comprising them, and their use for the treatment or prevention of infection by hepatitis C virus.

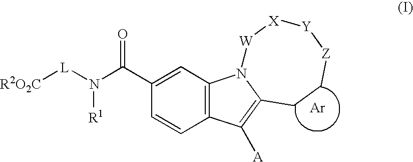

### CROSS-REFERENCE TO RELATED APPLICATIONS

This application is a National Stage of International Patent Application No. PCT/GB2006/050267, which was filed on Sep. 1, 2006. This application also claims the benefit of priority to British Provisional Patent Application No. GB 0518390.0, which was filed on Sep. 9, 2005.

### FIELD OF THE INVENTION

The present invention relates to tetracyclic indole compounds, to pharmaceutical compositions containing them, to their use in the prevention and treatment of hepatitis C infections and to methods of preparation of such compounds and compositions.

### BACKGROUND OF THE INVENTION

Hepatitis C (HCV) is a cause of viral infections. There is as yet no adequate treatment for HCV infection but it is believed that inhibition of its RNA polymerase in mammals, particularly humans, would be of benefit.

International patent applications publications WO 2003/010140 and WO 2004/065367 (both Boehringer Ingelheim) suggest indole derivatives as possible inhibitors of HCV polymerase. However, tetracyclic indole derivatives are not disclosed.

### SUMMARY OF THE INVENTION

Thus, the present invention provides the compound of the formula (I):

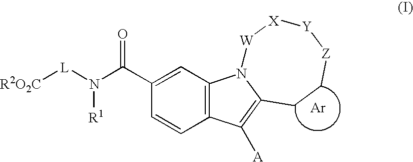

wherein

Ar is a moiety containing at least one aromatic ring and possesses 5-, 6-, 9- or 10-ring atoms optionally containing 1, 2 or 3 heteroatoms independently selected from N, O and S, which ring is optionally substituted by groups Q¹ and Q²;

Q¹ is halogen, hydroxy, C₁₋₆alkyl, C₁₋₆alkoxy, aryl, heteroaryl, CONR^{a}R^{b}, (CH₂)₀₋₃NR^{a}R^{b}, O(CH₂)₁₋₃NR^{a}R^{b}, O(CH₂)₀₋₃ CONR^{a}R^{b}, O(CH₂)₀₋₃aryl, O(CH₂)₀₋₃heteroaryl, OCHR^{c}R^{d};

R^{a} and R^{b} are each independently selected from hydrogen, C₁₋₄alkyl and C(O)C₁₋₄alkyl;

or R^{a}, R^{b} and the nitrogen atom to which they are attached form a heteroaliphatic ring of 4 to 7 ring atoms, where said ring is optionally substituted by halogen, hydroxy, C₁₋₄alkyl or C₁₋₄alkoxy;

R^{c} and R^{d} are each independently selected from hydrogen and C₁₋₄alkoxy;

or R^{c} and R^{d} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;

and wherein said C₁₋₄alkyl, C₁₋₄alkoxy and aryl groups are optionally substituted by halogen or hydroxy;

Q² is halogen, hydroxy, C₁₋₄alkyl or C₁₋₄alkoxy, where said C₁₋₄alkyl and C₁₋₄alkoxy groups are optionally substituted by halogen or hydroxy;

or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄alkoxy;

A is C₃₋₆alkyl or C₂₋₆alkenyl,

or A is a non-aromatic ring of 3 to 8 ring atoms where said ring may contain a double bond and/or may contain a O, S, SO, SO₂ or NH moiety,

or A is a non-aromatic bicyclic moiety of 4 to 8 ring atoms,

and A is optionally substituted by halogen, hydroxy, C₁₋₄alkyl or C₁₋₄alkoxy;

R¹ is hydrogen, C₁₋₆alkyl or C₂₋₆alkenyl;

R² is hydrogen or C₁₋₆alkyl;

L is

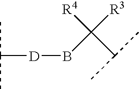

wherein R³ and R⁴ are each independently selected from hydrogen, halogen, C₁₋₄alkyl, C₂₋₄alkenyl or C₁₋₄alkoxy;

or R³ and R⁴ are linked to form a C₃₋₈cycloalkyl group;

B is aryl, heteroaryl, CONR⁵R⁶, optionally substituted by halogen, C₁₋₄alkyl, C₂₋₄alkenyl or C₁₋₄alkoxy;

R⁵ is hydrogen or C₁₋₆alkyl;

or R⁵ is linked to R³ and/or R⁴ to form a 5- to 10-membered ring, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;

R⁶ is aryl or heteroaryl;

or R⁵, R⁶ and the nitrogen atom to which they are attached form a 5- to 10-membered mono- or bi-cyclic ring system, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;

D is a bond, C₁₋₆alkylene, C₂₋₆alkenylene, C₂₋₆alkynylene, aryl or heteroaryl, where said aryl or heteroaryl is optionally substituted by halogen, C₁₋₄alkyl or C₂₋₄alkenyl;

W and Z are independently selected from a bond, C═O, O, S(O)₀₋₂, —(CR¹⁰R¹¹)—(CR¹²R¹³)₀₋₁— and NR¹⁰;

X and Y are independently selected from a bond, C═O, O, —CR¹⁴R¹⁵— and NR¹⁴;

and none, one or two of W, X, Y and Z are a bond;

R¹⁰, R¹¹, R¹², R¹³, R¹⁴ and R¹⁵ are each independently selected from hydrogen, hydroxy, C₁₋₆alkyl, C₂₋₆alkenyl, C₁₋₆alkoxy, C(O)C₁₋₆alkyl, Het, (CH₂)₀₋₃NR¹⁶R¹⁷, C(O)(CH₂)₀₋₃NR¹⁶R¹⁷, NHC(O)(CH₂)₀₋₃NR¹⁶R¹⁷, O(CH₂)₀₋₃NR¹⁶R¹⁷, S(O)₀₋₂(CH₂)₀₋₃R¹⁶R¹⁷ and C(O)(CH₂)₀₋₃OR¹⁶;

Het is a heteroaliphatic ring of 4 to 7 ring atoms, which ring may contain 1, 2 or 3 heteroatoms selected from N, O or S or a group S(O), S(O)₂, NH or NC₁₋₄alkyl;

R¹⁶ and R¹⁷ are independently selected from hydrogen, C₁₋₆alkyl and (CH₂)₀₋₄NR¹⁸R¹⁹;

or R¹⁶, R¹⁷ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;

R¹⁸ and R¹⁹ are independently selected from hydrogen and C₁₋₆alkyl;

or R¹⁸, R¹⁹ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;
and pharmaceutically acceptable salts thereof.

### DETAILED DESCRIPTION OF THE INVENTION

In one embodiment of the present invention, Ar is a 5- or 6-membered aromatic ring optionally containing 1, 2 or 3 heteroatoms independently selected from N, O and S, and which ring is optionally substituted by groups Q¹ and Q² as hereinbefore defined.

Preferably, Ar is a 5- or 6-membered aromatic ring optionally containing 1 or 2 heteroatoms independently selected from N, O or S, such as phenyl, pyridyl, pyridazinyl, pyrimidinyl, pyrazinyl, furanyl, pyrazolyl, imidazolyl and thienyl, which ring is optionally substituted by groups Q¹ and Q² as hereinbefore defined. More preferably, Ar is phenyl, 2-pyridyl, 3-pyridyl, 4-pyridyl, 2-furanyl or 3-furanyl, particularly phenyl, optionally substituted by groups Q¹ and Q² as hereinbefore defined.

Preferably, Q¹ is halogen, hydroxy, C₁₋₄alkyl, C₁₋₄alkoxy or (CH₂)₀₋₃N(C₁₋₆alkyl)₂. More preferably, Q¹ is fluorine, chlorine, methyl, methoxy or CH₂NMe₂. Most preferably, Q¹ is methoxy.

Preferably, Q² is absent.

In a further embodiment, A is C₃₋₆alkyl, C₂₋₆alkenyl or C₃₋₈cycloalkyl, where A is optionally substituted by halogen, hydroxy, C₁₋₄alkyl or C₁₋₄alkoxy. Preferably, A is C₃₋₈cycloalkyl, more preferably cyclopentyl or cyclohexyl, most preferably cyclohexyl, optionally substituted by halogen, hydroxy, C₁₋₄alkyl or C₁₋₄alkoxy.

Preferably, A is unsubstituted or substituted by fluorine, chlorine, methyl or methoxy, particularly fluorine. More preferably, A is unsubstituted.

In a further embodiment, R¹ is hydrogen or C₁₋₄alkyl. Preferably, R¹ is hydrogen or methyl. More preferably, R¹ is hydrogen.

In a further embodiment, R² is hydrogen or C₁₋₄alkyl. Preferably, R² is hydrogen or methyl. More preferably, R² is hydrogen.

In a further embodiment, R³ and R⁴ are linked to form a cyclobutyl, cyclopentyl or cyclohexyl group. Preferably, R³ and R⁴ are linked to form a cyclopentyl group.

In a further embodiment, B is CONR⁵aryl, optionally substituted by halogen, C₁₋₄alkoxy, where R⁵ is as hereinbefore defined. Preferably, B is CONHphenyl.

In a further embodiment, D is a bond or ethenylene. Preferably, D is ethenylene.

Examples of suitable L groups include:

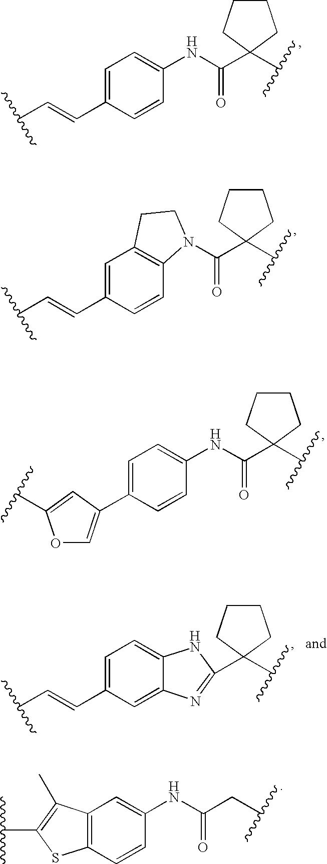

In a further embodiment, W is a bond, C═O, —(CR¹⁰R¹¹)—(CR¹²R¹³)₀₋₁— or NR¹⁰ where R¹⁰, R¹¹, R¹² and R¹³ are as hereinbefore defined. Preferably, W is —(CR¹⁰R¹¹)—(CR¹²R¹³)₀₋₁—, such as —CH₂—, —CH₂CH₂—, —CH(CH₃)—, —CH(CH₃)—CH(CH₃)—, —C(CH₃)₂— or —C(CH₃)₂—C(CH₃)₂—. More preferably, W is —CH₂— or —CH₂CH₂—. Most preferably, W is —CH₂—.

In a further embodiment, Z is a bond, C═O, —(CR¹⁰R¹¹)—(CR¹²R¹³)₀₋₁— or NR¹⁰ where R¹⁰, R¹¹, R¹² and R¹³ are as hereinbefore defined. Preferably, Z is a bond, O or —(CR¹⁰R¹¹)—(CR¹²R¹³)₀₋₁. More preferably, Z is a bond, O, —CH₂— or —CH₂CH₂—. Most preferably, Z is a bond, O or —CH₂—.

In a further embodiment, X is C═O, —CR¹⁴R¹⁵— or NR¹⁴ where R¹⁴ and R¹⁵ are as hereinbefore defined. Preferably, X is C═O, —CH₂—, —CH(C₁₋₆alkyl)-, —CHNHR¹⁶ or —CHN(CH₃)R¹⁶ where R¹⁶ is as hereinbefore defined. More preferably, X is C═O, —CH₂—, —CHNH—CH₂—CH₂—NR¹⁸R¹⁹ or —CHN(CH₃)—CH₂—CH₂—NR¹⁸R¹⁹ where R¹⁸ and R¹⁹ are as hereinbefore defined. Most preferably, X is —CH₂— or —CHN(CH₃)—CH₂—CH₂—N(CH₃)₂.

In a further embodiment, Y is C═O, —CR¹⁴R¹⁵— or NR¹⁴ where R¹⁴ and R¹⁵ are as hereinbefore defined. Preferably, Y is O, —CR¹⁴R¹⁵— or NR¹⁴. More preferably, Y is —CH₂—, —NH, N(C₁₋₆alkyl), NCH₂CH₂N(C₁₋₆alkyl)₂ or NHC(O)(CH₂)₁₋₂N(C₁₋₆alkyl)₂. Most preferably, Y is —CH₂—, NH, N(C₁₋₄alkyl), N(CH₂)₂N(C₁₋₄alkyl)₂ or NHC(O)CH₂N(C₁₋₄alkyl)₂. Especially, Y is —CH₂—, NCH₃ or N(CH₂)₂N(CH₃)₂.

In one embodiment of the present invention, there is provided the compound of formula (Ia):

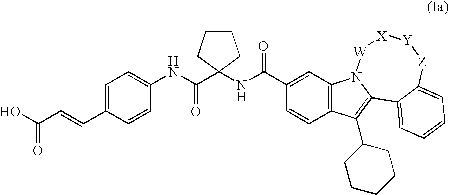

or a pharmaceutically acceptable salt thereof, wherein W, X, Y and Z are as defined in relation to formula (I).

Preferably, W is —CH₂— or —CH₂CH₂—. More preferably, W is —CH₂—.

Preferably, Z is a bond, O, —CH₂— or —CH₂CH₂—. More preferably, Z is a bond, O or —CH₂—.

Preferably, X is C═O, —CH₂—, —CH₂CH₂— or —CHN(C₁₋₄alkyl)-(CH₂)₂—N(C₁₋₄alkyl)₂. More preferably, X is —CH₂— or —CHN(CH₃)—(CH₂)₂—N(CH₃)₂.

Preferably, Y is —CR¹⁴R¹⁵— or NR¹⁴ where R¹⁴ and R¹⁵ are as hereinbefore defined. More preferably, Y is —CH₂— or NR¹⁴ where R¹⁴ is hydrogen, C₁₋₆alkyl or (CH₂)₀₋₃NR¹⁶R¹⁷ where R¹⁶ and R¹⁷ are as hereinbefore defined. Most preferably, Y is —CH₂—, NH, N(C₁₋₄alkyl) or N(CH₂)₂N(C₁₋₄alkyl)₂. Especially, Y is —CH₂—, NCH₃ or N(CH₂)₂N(CH₃)₂.

When any variable occurs more than one time in formula (I) or in any substituent, its definition on each occurrence is independent of its definition at every other occurrence.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy. Herein, “i-” indicates “iso-”, “i-” indicates “sec-”, and “t-” or “^{t}” indicated “tert-”.

The cycloalkyl groups referred to herein may represent, for example, cyclopropyl, cyclobutyl, cyclopentyl or cyclohexyl.

As used herein, the term “alkenyl” and “alkynyl” as a group or part of a group means that the group is straight or branched. Examples of suitable alkenyl groups include vinyl and allyl. A suitable alkynyl group is propargyl.

As used herein, the term “alkylene” means that the alkyl group links two separate groups and may be straight or branched. Examples of suitable alkylene groups include ethylene [—CH₂—CH₂—] and propylene [—CH₂—CH₂—CH₂—, —CH(CH₃)—CH₂— or —CH₂—CH(CH₃)—]. The terms “alkenylene” and “alkynylene” shall be construed in an analogous manner.

When used herein, the term “halogen” means fluorine, chlorine, bromine and iodine.

When used herein, the term “aryl” as a group or part of a group means a carbocyclic aromatic ring. Examples of suitable aryl groups include phenyl and naphthyl.

When used herein, the term “heteroaryl” as a group or part of a group means a 5- to 10-membered heteroaromatic ring system containing 1 to 4 heteroatoms selected from N, O and S. Particular examples of such groups include pyrrolyl, furanyl, thienyl, pyridyl, pyrazolyl, imidazolyl, oxazolyl, isoxazolyl, thiazolyl, isothiazolyl, pyrazinyl, pyrimidinyl, pyridazinyl, triazolyl, oxadiazolyl, thiadiazolyl, triazinyl, tetrazolyl, indolyl, benzothienyl, benzimidazolyl, benzofuryl, quinolinyl and isoquinolinyl.

Where a compound or group is described as “optionally substituted” one or more substituents may be present. Furthermore, optional substituents may be attached to the compounds or groups which they substitute in a variety of ways, either directly or through a connecting group. Particular examples of such connecting groups include amine, amide, ester, ether, thioether, sulfonamide, sulfamide, sulfoxide, urea, thiourea and urethane. As appropriate an optional substituent may itself be substituted by another substituent, the latter being connected directly to the former or through a connecting group such as those exemplified above.

Specific compounds within the scope of this invention include:
(2E)-3-(4-{[(1-{[(14-cyclohexyl-3-methoxy-6-methyl-5,6,7,8-tetrahydroindolo[2,1-a][2,5]benzodiazocin-11-yl)carbonyl]amino}cyclopentyl)carbonyl]amino}phenyl)acrylic acid,
(2E)-3-(4-{[(1-{[(14-cyclohexyl-6-methyl-5,6,7,8-tetrahydroindolo[2,1-a][2,5]benzodiazocin-11-yl)carbonyl]amino}cyclopentyl)carbonyl]amino}phenyl)acrylic acid,
(2E)-3-{4-[({1-[({13-cyclohexyl-5-[2-(dimethylamino)ethyl]-6,7-dihydro-5H-indolo[1,2-d][1,4]benzodiazepin-10-yl}carbonyl)amino]cyclopentyl}carbonyl)amino]phenyl}acrylic acid,
(2E)-3-{4-[({1-[({14-cyclohexyl-6-[2-(dimethylamino)ethyl]-5,6,7,8-tetrahydroindolo[2,1-a][2,5]benzodiazocin-11-yl}carbonyl)amino]cyclopentyl}carbonyl)amino]phenyl}acrylic acid, (2E)-3-{4-[({1-[({(7R)-[4-cyclohexyl-7-[[2-(dimethylamino)ethyl](methyl)amino]-7,8-dihydro-6H-indolo[1,2-e][1,5]benzoxazocin-11-yl}carbonyl)amino]cyclopentyl}carbonyl)amino]phenyl}acrylic acid
   and pharmaceutically acceptable salts thereof.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulfonic acid, maleic acid, succinic acid, acetic acid, citric acid, tartaric acid, carbonic acid, phosphoric acid or sulfuric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulfate ester, or reduction or oxidation of a susceptible functionality.

The present invention includes within its scope solvates of the compounds of formula (I) and slats thereof, for example, hydrates.

The present invention also includes within its scope any enantiomers, diastereomers, geometric isomers and tautomers of the compounds of formula (I). It is to be understood that all such isomers and mixtures thereof are encompassed within the scope of the invention.

The present invention further provides a compound of formula (I) or a pharmaceutically acceptable salt thereof for use in therapy.

In another aspect, the invention provides the use of a compound of formula (I) as defined above, or a pharmaceutically acceptable salt thereof, for the manufacture of a medicament for treatment or prevention of infection by hepatitis C virus in a human or animal.

A further aspect of the invention provides a pharmaceutical composition comprising a compound of formula (I) as defined above, or a pharmaceutically acceptable salt thereof, in association with a pharmaceutically acceptable carrier. The composition may be in any suitable form, depending on the intended method of administration. It may for example be in the form of a tablet, capsule or liquid for oral administration, or of a solution or suspension for administration parenterally.

The pharmaceutical compositions optionally also include one or more other agents for the treatment of viral infections such as an antiviral agent, or an immunomodulatory agent such as α-, β- or γ-interferon.

In a further aspect, the invention provides a method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the pharmaceutical composition described above or of a compound of formula (I) as defined above, or a pharmaceutically acceptable salt thereof. “Effective amount” means an amount sufficient to cause a benefit to the subject or at least to cause a change in the subject's condition.

The dosage rate at which the compound is administered will depend on a variety of factors including the activity of the specific compound employed, the metabolic stability and length of action of that compound, the age of the patient, body weight, general health, sex, diet, mode and time of administration, rate of excretion, drug combination, the severity of the particular condition and the host undergoing therapy. Suitable dosage levels may be of the order of 0.02 to 5 or 10 g per day, with oral dosages two to five times higher. For instance, administration of from 1 to 50 mg of the compound per kg of body weight from one to three times per day may be in order. Appropriate values are selectable by routine testing. The compound may be administered alone or in combination with other treatments, either simultaneously or sequentially. For instance, it may be administered in combination with effective amounts of antiviral agents, immunomodulators, anti-infectives or vaccines known to those of ordinary skill in the art. It may be administered by any suitable route, including orally, intravenously, cutaneously and subcutaneously. It may be administered directly to a suitable site or in a manner in which it targets a particular site, such as a certain type of cell. Suitable targeting methods are already known.

An additional aspect of the invention provides a method of preparation of a pharmaceutical composition, involving admixing at least one compound of formula (I) as defined above, or a pharmaceutically acceptable salt thereof, with one or more pharmaceutically acceptable adjuvants, diluents or carriers and/or with one or more other therapeutically or prophylactically active agents.

The present invention also provides a process for the preparation of compounds of formula (I).

According to a general process (a), compounds of formula (I) may be prepared by the reaction of a compound of formula (II) with a compound of formula (III):

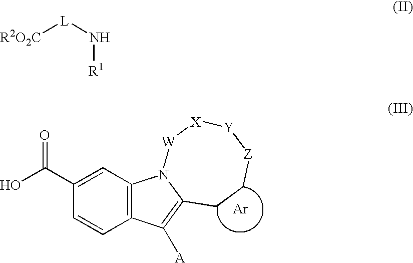

where R¹, R², L, A, Ar, W, X, Y and Z are as defined in relation to formula (I). The reaction may conveniently be carried out in the presence of a coupling reagent, such as HATU, and a base, such as diisopropylethylamine, in a suitable solvent, such as DMF.

Compounds of formulae (II) and (III) are either known in the art or may be prepared by conventional methodology well known to one of ordinary skill in the art using, for instance, procedures described in the accompanying Descriptions and Examples, or by alternative procedures which will be readily apparent.

For example, compounds of formula (III) may be prepared by internal ring closure of the compound of formula (IV):

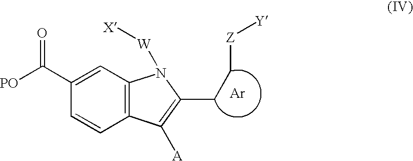

where A, Ar, W and Z are as defined in relation to formula (I), P is a suitable protecting group, such as methyl, and X and Y have suitable precursor functionality to either or both of groups X and Y as defined in relation to formula (I). For instance, when X is —CH₂— and Y is N(CH₃), X′ and Y′ can be —CHO and —NC(CH₃) respectively, where the reaction is carried out in the presence of a mild reducing agent, such as sodium cyanoborohydride, under mild acidic conditions in a suitable solvent, such as methanol. Alternatively, when X is C═O and Y is NH, X′ and Y′ can be C(O)O^{t}Bu and NHC(O)O^{t}Bu respectively, where the reaction is carried out under acidic conditions in a suitable solvent system, such as a dichloromethane/water mixture.

Compounds of formula (I) can be converted into other compounds of formula (I) using synthetic methodology well known in the art. For instance, the compound of formula (I) where R¹ is CO₂CH₂CH₃ may be converted into the compound of formula (I) where R¹ is CO₂H by conversion of the ester to the carboxylic acid, for example, by treatment with LiOH in a suitable solvent, such as dioxane, THF and/or methanol in the presence of water.

In addition, the compound of formula (I) where X is C═O may be converted into the compound of formula (I) where X is CH₂ by reduction of the oxo group with, for instance, a borane reagent, such as BH₃.Me₂S, in a suitable solvent, such as THF.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3rd edition, 1999. The protecting groups may be removed at a convenient subsequent stage using methods known from the art.

The present invention is illustrated further by the following non-limiting examples.

The compounds of the invention were tested for inhibitory activity against the HCV RNA dependent RNA polymerase (NS5B) in an enzyme inhibition assay (example (i)) and in a cell-based sub-genomic replication assay (example (ii)). The compounds generally have IC50's below 1 μM in the enzyme assay and several examples have EC50's below 0.5 μM in the cell based assay.

Compound names in the examples were generated using software from ACDLabs (version 6.0).

### (i) In-Vitro HCV NS5B Enzyme Inhibition Assay

WO 96/37619 describes the production of recombinant HCV RdRp from insect cells infected with recombinant baculovirus encoding the enzyme. The purified enzyme was shown to possess in vitro RNA polymerase activity using RNA as template. The reference describes a polymerization assay using poly(A) and oligo(U) as a primer or an heteropolymeric template. Incorporation of tritiated UTP or NTPs is quantified by measuring acid-insoluble radioactivity. The present inventors have employed this assay to screen the various compounds described above as inhibitors of HCV RdRp.

Incorporation of radioactive UMP was measured as follows. The standard reaction (50 μl) was carried out in a buffer containing 20 mM tris/HCl pH 7.5, 5 mM MgCl₂, 1 mM DTT, 50 mM NaCl, 0.03% N-octylglucoside, 1 μCi [³H]-UTP (40 Ci/mmol, NEN), 10 μM UTP and 10 μg/ml poly(A) or 5 μM NTPs and 5 μg/ml heteropolymeric template. Oligo(U)₁₂ (1 μg/ml, GENSET) was added as a primer in the assay working on Poly(A) template. The final NS5B enzyme concentration was 5 nM. The order of assembly was: 1) compound, 2) enzyme, 3) template/primer, 4) NTP. After 1 h incubation at 22° C. the reaction was stopped by adding 50 μl of 20% TCA and applying samples to DE81 filters. The filters were washed thoroughly with 5% TCA containing 1M Na₂HPO₄/NaH₂PO₄, pH 7.0, rinsed with water and then ethanol, air dried, and the filter-bound radioactivity was measured in the scintillation counter. Carrying out this reaction in the presence of various concentrations of each compound set out above allowed determination of IC₅₀ values by utilizing the formula:

% Residual activity=100/(1+[I]/IC₅₀)^{S}

where [I] is the inhibitor concentration and “s” is the slope of the inhibition curve.

### (ii) Cell-Based HCV Replication Assay

Cell clones that stably maintain subgenomic HCV replicon were obtained by transfecting Huh-7 cells with an RNA replicon identical to I₃₇₇neo/NS3-3′/wt described by V. Lohmann et al., 285 SCIENCE 110 (Jul. 2, 1999) (EMBL-GENBANK No. AJ242652), followed by selection with neomycin sulfate (G418). Viral replication was monitored by measuring the expression of the NS3 protein by an ELISA assay performed directly on cells grown in 96-well microtiter plates (Cell-ELISA) using the anti-NS3 monoclonal antibody 10E5/24 (as described in International patent application publication WO 02/59321). Cells were seeded into 96-well plates at a density of 10⁴ cells per well in a final volume of 0.1 ml of DMEM/10% FCS. Two hours after plating, 50 μl of DMEM/10% FCS containing a 3× concentration of inhibitor were added, cells were incubated for 96 hours and then fixed for 10′ with ice-cold isopropanol. Each condition was tested in duplicate and average absorbance values were used for calculations. The cells were washed twice with PBS, blocked with 5% non-fat dry milk in PBS+0.1% TRITON X100+0.02% SDS (PBSTS) and then incubated o/n at 4° C. with the 10E5/24 mab diluted in Milk/PBSTS. After washing 5 times with PBSTS, the cells were incubated for 3 hours at room temperature with Fc specific anti-mouse IgG conjugated to alkaline phosphatase (Sigma), diluted in Milk/PBSTS. After washing again as above, the reaction was developed with p-Nitrophenyl phosphate disodium substrate (Sigma) and the absorbance at 405/620 nm read at intervals. For calculations, data sets where samples incubated without inhibitors had absorbance values comprised between 1 and 1.5 were used. The inhibitor concentration that reduced by 50% the expression of NS3 (IC₅₀) was calculated by fitting the data to the Hill equation,

Fraction inhibition=1−(Ai−b)/(A₀−b)=[I]ⁿ/([I]ⁿ+IC₅₀)

where:
Ai=absorbance value of HBI10 cells supplemented with the indicated inhibitor concentration.
A₀=absorbance value of HBI10 cells incubated without inhibitor.
b=absorbance value of Huh-7 cells plated at the same density in the same microtiter plates and incubated without inhibitor.
n=Hill coefficient.
   (iii) General Procedures

All solvents were obtained from commercial sources (FLUKA, PURISS.) and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporators operating under reduced pressure. Flash chromatography was carried out on silica gel following published procedure (W. Clark Still et al., Rapid Chromatographic Technique for Preparative Separations with Moderate Resolution, 43(14) J. ORG. CHEM. 2923-25 (1978)) or on commercial flash chromatography systems (BIOTAGE corporation and Jones FLASHMASTER II) utilizing pre-packed columns.

Reagents were usually obtained directly from commercial suppliers (and used as supplied) but a limited number of compounds from in-house corporate collections were utilized. In the latter case the reagents are readily accessible using routine synthetic steps that are either reported in the scientific literature or are known to those skilled in the art.

¹H NMR spectra were recorded on BRUKER AM series spectrometers operating at (reported) frequencies between 300 and 600 MHz. Chemical shifts (6) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet; q, quartet; m, multiplet; br, broad, and combinations thereof); coupling constant(s) in hertz (Hz); number of protons. Mass spectral (MS) data were obtained on a PERKIN ELMER API 100, or WATERS MICROMASS ZQ, operating in negative (ES⁻) or positive (ES⁺) ionization mode and results are reported as the ratio of mass over charge (m/z) for the parent ion only. Preparative scale HPLC separations were carried out on a WATERS DELTA PREP 4000 separation module, equipped with a WATERS 486 absorption detector or on a GILSON preparative system. In all cases compounds were eluted with linear gradients of water and acetonitrile both containing 0.1% TFA using flow rates between 15 and 40 mL/min.

The following abbreviations are used in the examples, the schemes and the tables: Ar: aryl; cat.: catalytic; DCM: dichloromethane; dioxan(e): 1,4-dioxane; DIPEA: diisopropylethyl amine; DMAP: N,N-dimethylpyridin-4-amine; DME: dimethoxyethane; DMF: dimethylformamide; DMSO: dimethylsulfoxide; EDAC.HCl: 1-ethyl-(3-dimethylaminopropyl)carbodiimide HCl salt; eq.: equivalent(s); Et: ethyl; EtOAc: ethyl acetate; Et₂O: diethyl ether; EtOH: ethanol; h: hour(s); HATU: O-(7-azabenzotriazol-1-yl)-N,N,N′,N′-tetramethyluronium hexafluorophophate; Me: methyl; MeCN: acetonitrile; MeOH: methanol; min: minutes; MS: mass spectrum; NBS: N-bromo succinimide; PE: petroleum ether; Ph: phenyl; Prep.: preparative; ⁱPr₂NEt: diisopropylethyl amine; quant.: quantitative; RP-HPLC: reversed phase high-pressure liquid chromatography; RT/rt: room temperature; sol.: solution; TFA: trifluoroacetic acid; THF: tetrahydrofuran; TMS: trimethylsilyl.

### Description 1: 14-cyclohexyl-3-methoxy-6-methyl-5,6,7,8-tetrahydroindolo[2,1-a][2,5]benzodiazocine-11-carboxylic Acid

### Step 1: methyl 2-bromo-3-cyclohexyl-1-(1,3-dioxolan-2-ylmethyl)-1H-indole-6-carboxylate

NaH (1.5 eq., 60% dispersion in mineral oil) was added to a solution of methyl 2-bromo-3-cyclohexyl-1H-indole-6-carboxylate (prepared as described in International patent application publication WO 2004/065367) in DMF (0.1 M) and once effervescence had subsided the solution was allowed to stir at RT for a further 30 min. 2-bromomethyl-1,3-dioxolane (4 eq.) and catalytic (0.025 eq.) potassium iodide were then added and the mixture heated at 50° C. for 36 h. The reaction mixture was allowed to cool to RT, quenched with aqueous HCl (1 N) and extracted with EtOAc. The organics were washed with aqueous HCl (1 N) (3×), water and brine before being dried over Na₂SO₄, filtered and the solvent evaporated in vacuo. Purification was by flash chromatography (10% EtOAc/PE) to give a pale yellow solid that was triturated with Et₂O/PE to afford the title compound as a white solid (69%). ¹H NMR (400 MHz, DMSO-d₆, 300 K) δ 1.35-1.45 (m, 3H), 1.68-1.80 (m, 3H), 1.8-2.0 (m, 4H), 2.83-2.89 (m, 1H), 3.78 (s, 4H), 3.88 (s, 3H), 4.45-4.46 (m, 2H), 5.13-5.18 (m, 1H) 7.65 (d, J 8.5, 1H), 7.81 (d, J 8.5, 1H), 8.14 (s, 1H); MS (ES⁺) m/z 422 (M+H)⁺, m/z 424 (M+H)⁺

### Step 2: methyl 3-cyclohexyl-1-(1,3-dioxolan-2-ylmethyl)-2-(2-formyl-4-methoxyphenyl)-1H-indole-6-carboxylate

To a solution of methyl 2-bromo-3-cyclohexyl-1-(1,3-dioxolan-2-ylmethyl)-1H-indole-6-carboxylate (from Step 1) in dioxane (0.1 M) were added Na₂CO₃ (6 eq., 2 M aqueous solution), 4-methoxy-2-formylphenylboronic acid (2 eq.) and bis(triphenylphosphine)palladium(II) dichloride (0.2 eq.). The mixture was degassed before being heated at reflux for 30 min. RP-HPLC analysis of the reaction mixture showed starting material persisted. The reaction mixture was allowed to cool and an additional 1 eq. of 4-methoxy-2-formylphenylboronic acid and 0.1 eq. of bis(triphenylphosphine)palladium(II) dichloride introduced. Heating at reflux was then resumed for a further 30 min. The reaction was allowed to cool to RT and partitioned between water and EtOAc. The aqueous fraction was extracted with EtOAc and the combined organics washed with aqueous HCl (1 N), water and brine before being dried over Na₂SO₄, filtered and concentrated in vacuo. The crude material was purified by flash chromatography (10-20% gradient EtOAc/PE) to afford the title compound as a yellow foam (72%). ¹H NMR (400 MHz, DMSO-d₆, 300 K) δ 1.10-1.24 (m, 3H), 1.60-1.80 (m, 7H), 2.30-2.39 (m, 1H), 3.41-3.48 (m, 1H), 3.56-3.65 (m, 3H), 3.89 (s, 3H), 3.94 (s, 3H), 3.98 (dd, J 15.3, 4.4, 1H), 4.25 (dd, J 15.3, 2.6, 1H), 4.92-4.93 (m, 1H), 7.40-7.46 (m, 2H), 7.49 (d, J 2.2, 1H), 7.70 (d, J 8.8, 1H), 7.85 (d, J 8.8, 1H), 8.21 (s, 1H), 9.61 (s, 1H); MS (ES⁺) m/z 478 (M+H)⁺

### Step 3: methyl 3-cyclohexyl-1-(1,3-dioxolan-2-ylmethyl)-2-{4-methoxy-2-[(methylamino)methyl]phenyl}-1H-indole-6-carboxylate

To a solution of methyl 3-cyclohexyl-1-(1,3-dioxolan-2-ylmethyl)-2-(2-formyl-4-methoxyphenyl)-1H-indole-6-carboxylate (from Step 2) in THF (0.05 M), methylamine (10 eq., 2 M solution in THF) was added and the pH adjusted to pH 6 with acetic acid. The solution was stirred at RT for 45 min before being concentrated in vacuo. The residue was taken up in MeOH to give a 0.025 M solution. NaBH₃CN (2.4 eq.) were added and the mixture stirred at RT for 2 h. RP-HPLC analysis of the reaction mixture showed the complete conversion to the desired amine. The reaction was quenched with saturated aqueous NaHCO₃ and extracted (2×) with EtOAc. The combined organics were washed with water and brine before being dried over Na₂SO₄, filtered and concentrated in vacuo to give the title compound as a viscous oil (89%). ¹H NMR (400 MHz, DMSO-d₆, 300 K) [symbol] 1.10-1.27 (m, 3H), 1.60-1.75 (m, 6H), 1.79-1.90 (m, 1H), 2.20 (s, 3H), 2.29-2.35 (m, 1H), 3.34 (br s, 2H, partially obscured by water peak), 3.63-3.72 (m, 3H), 3.73-3.78 (m, 1H), 3.79-3.84 (m, 1H), 3.85 (s, 3H), 3.87 (s, 3H), 4.15 (dd, J 15.0, 4.7, 1H), 4.84-4.87 (m, 1H), 6.96 (dd, J 8.5, 2.6, 1H), 7.18 (d, J 8.5, 1H), 7.23 (d, J 2.6, 1H), 7.65 (dd, J 8.4, 1.3, 1H), 7.80 (d, J 8.4, 1H), 8.16 (d, J 1.3, 1H); MS (ES⁺) m/z 493 (M+H)⁺

### Step 4: methyl 3-cyclohexyl-2-{4-methoxy-2-[(methylamino)methyl]phenyl}-1-(2-oxoethyl)-1H-indole-6-carboxylate

Aqueous HCl (25 eq., 3 M) was added to a solution of methyl 3-cyclohexyl-1-(1,3-dioxolan-2-ylmethyl)-2-{4-methoxy-2-[(methylamino)methyl]phenyl}-1H-indole-6-carboxylate (from Step 3) in THF (0.02 M), and the mixture heated at reflux for 24 h. ¹H NMR analysis of an aliquot from the reaction mixture confirmed the complete conversion of starting material. The volatiles were reduced in vacuo, and the residue partitioned between EtOAc and saturated aqueous NaHCO₃ (ensuring that the aqueous phase was basic). The aqueous phase was extracted with EtOAc and the combined organics washed with water and brine, before being dried over Na₂SO₄, filtered and concentrated in vacuo to give the title compound in essentially quantitative yield. ¹H NMR (400 MHz, DMSO-d₆, 300 K) δ 1.05-1.35 (m, 3H), 1.60-1.95 (m, 7H), 2.25-2.33 (m, 1H), 2.86 (s, 3H), 3.74-3.78 (m, 1H, obscured by water peak), 3.88 (s, 3H), 3.95 (s, 3H), 4.29 (d, J 14.2, 1H), 5.31 (d, J 7.3, 1H), 5.70 (d, J 7.3, 1H), 7.06-7.10 (m, 2H), 7.32 (d, J 8.3, 1H), 7.63 (dd, J 8.4, 1.4, 1H), 7.84 (d, J 8.4, 1H), 7.96 (d, J 1.4, 1H), 9.02 (br s, 1H); MS (ES) m/z 449 (M+H)⁺

### Step 5: 14-cyclohexyl-3-methoxy-6-methyl-5,6,7,8-tetrahydroindolo[2,1-a][2,5]-benzodiazocine-11-carboxylic Acid

Acetic acid was added dropwise to a stirred solution of methyl 3-cyclohexyl-2-{4-methoxy-2-[(methylamino)methyl]phenyl}-1-(2-oxoethyl)-1H-indole-6-carboxylate (from Step 4) in MeOH (0.005 M) at RT, to adjust the pH to pH 6. The mixture was stirred for 10 min prior to introducing NaCNBH₃ (3.2 eq.). RP-HPLC analysis of the reaction mixture after 1 h confirmed the complete conversion of the aminoaldehyde to the desired cyclic amine. The reaction was diluted with an equal volume of THF and NaOH (100 eq., 2 M aqueous solution) introduced. The reaction mixture was then heated at 60° C. for 3 h before being allowed to cool to RT. The THF/MeOH volume was reduced in vacuo and the residue acidified with aqueous HCl (1 N) before being extracted with EtOAc (4×). The combined organics were washed with brine, dried over Na₂SO₄, filtered and concentrated in vacuo to afford the hydrochloride salt of the product as a yellow solid. Purification was by automated RP-MS-HPLC (stationary phase: column WATERS XTERRA prep. C18, 5 um, 19×100 mm. Mobile phase: MeCN/H₂O buffered with 0.1% TFA). Fractions containing the pure compound were combined and freeze dried to afford the title compound as a white powder (21%). ¹H NMR (400 MHz, DMSO-d₆, 300 K) δ 1.09-1.21 (m, 1H), 1.28-1.37 (m, 2H), 1.50-1.56 (m, 1H), 1.65-1.75 (m, 2H), 1.82-2.0 (m, 4H), 2.58-2.67 (m, 1H), 3.04 (br s, 3H), 3.3-3.5 (m, 1H, obscured by water peak), 3.63-3.75 (m, 3H), 3.91 (s, 3H), 4.32 (d, J 13.4, 1H), 4.79 (dd, J 16.0, 3.5, 1H), 7.25 (dd, J 8.5, 2.3, 1H), 7.40 (d, J 8.5, 1H), 7.61 (d, J 2.3, 1H), 7.73 (d, J 8.3, 1H), 7.91 (d, J 8.3, 1H), 8.19 (s, 1H), 9.86 (br s, 1H), 12.68 (br s, 1H); MS (ES⁺) m/z 419 (M+H)⁺

### Description 2: 14-cyclohexyl-6-[2-(dimethylamino)ethyl]-5,6,7,8-tetrahydroindolo[2,1-a][2,5]benzodiazocine-11-carboxylic Acid

### Step 1: Methyl 2-bromo-3-cyclohexyl-1-(2,2-dimethoxyethyl)-1H-indole-6-carboxylate

To a stirred solution of methyl 2-bromo-3-cyclohexyl-1H-indole-6-carboxylate (prepared as described in International patent application publication WO 2004/087714) (0.2 M, 1 eq.) in DMF at RT was added NaH (60% dispersion in mineral oil, 1.75 eq). After 1 h, KI (8 mol %) and bromoacetaldehyde dimethyl acetal (2.5 eq) were added and the reaction heated at 80° C. for 17 h., the reaction was quenched by addition of aqueous HCl (IN) and extracted into EtOAc (×3). The combined organics were washed with HCl (1N), H₂O and brine before being dried over Na₂SO₄, filtered and concentrated in vacuo. Purification by flash column chromatography (BIOTAGE, 5-10% EtOAc/PE gradient) gave the title compound as a white solid (79%). ¹H NMR (300 MHz, d₆-DMSO, 300 K) δ 1.51-1.68 (m, 3H), 1.67-1.72 (m, 3H), 1.82-1.99 (m, 4H), 2.81-2.89 (m, 1H), 3.25 (s, 6H), 3.87 (s, 3H), 4.35-4.37 (m, 2H), 4.46-4.59 (m, 1H), 7.64 (d, J 8.4, 1H), 7.80 (d, J 8.4, 1H), 8.10 (s, 1H); MS (ES⁺) m/z 446 (M+H)⁺, 448 (M+H)⁺

### Step 2: Methyl 3-cyclohexyl-1-(2,2-dimethoxyethyl)-2-(2-formylphenyl)-1H-indole-6-carboxylate

A solution of methyl 2-bromo-3-cyclohexyl-1-(2,2-dimethoxyethyl)-1H-indole-6-carboxylate (0.16 M, 1 eq, from Step 1) in dioxane and Na₂CO₃ (6 eq of a 2M solution) was degassed by sonication for 10 min. 2-Formylphenylboronic acid (1.5 eq) and bis(triphenylphosphine) palladium(II) dichloride (20 mol %) were added and the reaction placed in a pre-heated oil bath at 108° C. for 20 min until it went black. After cooling to RT, the reaction was partitioned between H₂O and EtOAc (×3). The combined organics were washed with HCl (1N), H₂O, and brine before being dried over Na₂SO₄, filtered and concentrated in vacuo. Purification by flash column chromatography (BIOTAGE, 10% EtOAc/PE) gave the title compound as a pale yellow solid (85%). ¹H NMR (300 MHz, d₆-DMSO, 300 K) δ 1.08-1.23 (m, 3H), 1.60-1.81 (m, 7H), 2.30-2.34 (m, 1H), 3.01 (s, 3H), 3.07 (s, 3H), 3.89 (s, 3H), 3.95-3.97 (m, 1H), 4.12-4.18 (m, 1H), 4.36-4.37 (m, 1H), 7.53 (d, J 7.5, 1H), 7.70-7.78 (m, 2H), 7.84-7.88 (m, 2H), 8.04 (d, J 7.5, 1H), 8.17 (s, 1H), 9.66 (s, 1H); MS (ES⁺) m/z 472 (M+Na)⁺, 450 (M+H)⁺

### Step 3: Methyl 3-cyclohexyl-1-(2,2-dimethoxyethyl)-2-[2-({[2-(dimethylamino)ethyl]amino}methyl)phenyl]-1H-indole-6-carboxylate

To a stirred solution of methyl 3-cyclohexyl-1-(2,2-dimethoxyethyl)-2-(2-formylphenyl)-1H-indole-6-carboxylate (0.16 M, 1 eq., from Step 2) and 2-dimethylaminoethylamine (2 eq.) in THF was added glacial acetic acid to adjust the pH of the reaction to circa pH 4. The reaction was stirred for 1 h after which the THF was removed under reduced pressure and the residue redissolved in MeOH. NaBH₄ (8 eq.) was added portion-wise until complete conversion was observed by LC-MS analysis. The reaction was quenched by addition of sat. aq. NaHCO₃ and extracted into EtOAc (×3). The combined organics were washed with H₂O and brine before being dried over Na₂SO₄, filtered and concentrated in vacuo. The title compound was obtained as a pale yellow oil and taken on without further purification (quantitative). ¹H NMR (300 MHz, d₆-DMSO, 300 K) δ 1.15-1.23 (m, 3H), 1.67-1.72 (m, 7H), 2.16 (s, 6H), 2.33-2.50 (m, 5H obscured by DMSO), 3.00 (s, 3H), 3.08 (s, 3H), 3.40-3.50 (m, 2H obscured by H₂O), 3.78 (dd, J 14.8, 4.6, 1H), 3.88 (s, 3H), 4.07-4.14 (m, 1H), 4.26-4.28 (m, 1H), 7.29 (d, J 7.5, 1H), 7.40-7.44 (m, 1H), 7.52-7.56 (m, 1H), 7.65-7.70 (m, 2H), 7.83 (d, J 8.4, 1H), 8.1 (s, 1H); MS (ES⁺) m/z 522 (M+H)⁺

### Step 4: Methyl 14-cyclohexyl-6-[2-(dimethylamino)ethyl]-5,6,7,8-tetrahydroindolo[2,1-a][2,5]-benzodiazocine-11-carboxylate

To a stirred solution of methyl 3-cyclohexyl-1-(2,2-dimethoxyethyl)-2-[2-({[2-(dimethylamino)ethyl]amino}methyl)phenyl]-1H-indole-6-carboxylate (0.16 M, 1 eq., from Step 3) in THF was added an equal volume of aqueous 1N HCl. The reaction was heated at 60° C. for 2.5 h and after cooling to RT was quenched by addition of NaOH (2N) and extracted into EtOAc (×3). The combined organic extracts were washed with brine before being dried over Na₂SO₄, filtered and concentrated in vacuo. The residue was re-dissolved in MeOH and acidified to pH 4 with glacial acetic acid. After stirring for 45 min, NaBH₄ (8 eq.) was added portion-wise until cyclization was complete as evidenced by LC-MS analysis. The reaction was quenched by addition of sat. aq. NaHCO₃ and extracted into EtOAc (×3). The combined organic extracts were washed with brine before being dried over Na₂SO₄, filtered and concentrated in vacuo. The title compound was obtained as a pale yellow oil and taken on without further purification (quantitative). ¹H NMR (400 MHz, d₆-DMSO, 300 K) δ 1.12-1.35 (m, 3H), 1.50-1.53 (m, 1H), 1.66-1.72 (m, 2H), 1.79-1.86 (m, 1H), 1.94-1.92 (m, 3H), 2.53-2.57 (m, 1H obscured by DMSO), 2.61-2.65 (m, 2H), 2.82 (s, 6H), 2.85-2.92 (m, 3H), 3.25-3.36 (m, 2H obscured by H₂O), 3.51-3.57 (m, 1H), 3.79 (d, J 13.8, 1H), 3.88 (s, 3H), 4.47-4.52 (m, 1H), 7.38 (d, J 7.5, 1H), 7.47-7.50 (m, 1H), 7.53-7.57 (m, 1H), 7.64 (d, J 7.5, 1H), 7.70 (d, J 8.4, 1H), 7.91 (d, J 8.4, 1H), 8.10 (s, 1H); MS (ES⁺) m/z 460 (M+H)⁺

### Step 5: 14-cyclohexyl-6-[2-(dimethylamino)ethyl]-5,6,7,8-tetrahydroindolo[2,1-a][2,5]-benzo diazocine-11-carboxylic Acid

A solution of methyl 14-cyclohexyl-6-[2-(dimethylamino)ethyl]-5,6,7,8-tetrahydroindolo[2,1-a][2,5]benzodiazocine-11-carboxylate (0.16 M, 1 eq, from Step 4) in MeOH and 1N NaOH (4 eq) was heated to 80° C. for 6 h. After cooling to RT, the MeOH was removed under reduced pressure and the resulting aqueous solution acidified with aqueous 3N HCl until pH 1-2 resulting in formation of a pale yellow precipitate. This was filtered off and dried on the filter overnight to afford the crude hydrochloride salt of the product as a yellow solid. Purification was by RP-HPLC (stationary phase: column WATERS XTERRA prep. MS C18, 5 μm, 30×100 mm. Mobile phase: MeCN/H₂O buffered with 0.1% TFA). Fractions containing the pure compound were combined and freeze dried (2×) in the presence of 3N aqueous HCl to afford the bis-HCl salt of the title compound as a white powder (65% over steps 3, 4 and 5). ¹H NMR (400 MHz, d₆-DMSO+TFA, 300K) δ 1.12-1.15 (m, 1H), 1.31-1.36 (m, 2H), 1.53-1.56 (m, 1H), 1.67-1.72 (m, 2H), 1.82-1.84 (m, 1H), 1.90-1.99 (m, 3H), 2.62-2.69 (m, 1H), 2.87 (s, 6H), 3.45-3.50 (m, 1H), 3.62-3.82 (m, 7H), 4.52 (d, J 13.6, 1H), 4.84 (dd, J 16.6, 4.6, 1H), 7.47-7.49 (m, 1H), 7.63-7.68 (m, 2H), 7.74 (d, J 8.4, 1H), 7.93-7.95 (m, 2H), 8.2 (s, 1H); MS (ES⁺) m/z 446 (M+H)⁺

### EXAMPLE 1

### (2E)-3-(4-{[(1-{[(14-cyclohexyl-3-methoxy-6-methyl-5,6,7,8-tetrahydroindolo[2,1-a][2,5]benzodiazocin-11-yl)carbonyl]amino}cyclopentyl)carbonyl]amino}phenyl)acrylic Acid

### Step 1: Ethyl (2E)-3-(4-{[(1-aminocyclopentyl)carbonyl]amino}phenyl)acrylate

1-{[(benzyloxy)carbonyl]amino}cyclopentanecarboxylic acid was dissolved in DMF (0.2 M). HATU (1 eq.) and triethylamine (3 eq.) were added, followed by ethyl (2E)-3-(4-aminophenyl)acrylate (0.95 eq.). The resulting mixture was stirred for 48 h at 40° C. DMF was evaporated, the resulting oil taken up in EtOAc and the solution washed with hydrochloric acid (3×, 1 M), water, a solution of saturated aqueous NaHCO₃ (2×) and brine. Drying over sodium sulfate and evaporation gave an orange solid, which was purified by flash chromatography on silica gel using PE/EtOAc (2.5:1, containing 1% EtOH) as the eluant. The resulting solid was immediately dissolved in DCM (0.1 M) and triflic acid (5 eq.) was added dropwise at RT. After 5 min at RT, the red mixture was poured into an aqueous solution of NaHCO₃. The organic phase was separated, the aqueous phase was extracted with DCM (4×) and the combined organic phases dried over sodium sulfate. Evaporation gave the title compound as an off-white solid, which was used without further purification. ¹H NMR (400 MHz, DMSO-d₆, 300 K) δ 1.26 (t, J 7.1, 3H), 1.48-1.61 (m, 2H), 1.63-1.87 (m, 4H), 1.96-2.10 (m, 2H), 4.188 (q, J 7.1 Hz, 2H), 6.53 (d, J 16.0, 1H), 6.60-7.30 (bs, 3H), 7.59 (d, J 16.0, 1H), 7.67 (d, J 8.5, 2H), 7.76 (d, J 8.5, 2H); MS (ES⁺) m/z 303 (M+H)⁺.

Ethyl (2E)-3 (4-{[(1-aminocyclopentyl)carbonyl]amino}phenyl)acrylate, as its HCl salt, was also prepared by coupling 1-[(tert-butoxycarbonyl)amino]cyclopentanecarboxylic acid to (2E)-3-(4-aminophenyl)acrylate in analogous fashion to that described above. Deprotection with HCl in EtOAc then afforded the HCl salt. Ethyl (2E)-3 (4-{[(1-aminocyclopentyl)carbonyl]amino}phenyl)acrylate, as free base or salt, was used interchangeably in subsequent couplings—simply employing an additional equivalent of base to neutralize the HCl salt as necessary.

### Step 2: Ethyl (2E)-3-(4-{[(1-{[(14-cyclohexyl-3-methoxy-6-methyl-5,6,7,8-tetrahydroindolo[2,1-a][2,5]-benzodiazocin-11-yl)carbonyl]amino}cyclopentyl)carbonyl]amino}phenyl)acrylate

HATU (1.1 eq) was added to a stirred solution of 14-cyclohexyl-3-methoxy-6-methyl-5,6,7,8-tetrahydroindolo[2,1-a][2,5]benzodiazocine-11-carboxylic acid (from Description 1, 0.015 M), the hydrochloride salt of ethyl (2E)-3-(4-{[(1-aminocyclopentyl)carbonyl]amino}phenyl)acrylate (from Step 1, 1.1 eq.) and ⁱPr₂NEt (3.5 eq.) in dry DMF. The reaction was then heated at 50° C. for 2 h. The reaction was allowed to cool to rt and a further 0.2 eq. HATU, 0.2 eq ethyl (2E)-3-(4-{[(1-aminocyclopentyl)carbonyl]amino}phenyl)acrylate HCl salt and 1 eq. ⁱPr₂NEt were introduced before resuming heating for 1.5 h. The reaction was allowed to cool to rt, quenched with saturated aqueous NaHCO₃ and extracted into EtOAc. The aqueous phase was extracted a second time with EtOAc and the combined organics washed with saturated aqueous NaHCO₃, water, 1N HCl (3×) and brine before being dried over Na₂SO₄, filtered and concentrated in vacuo to afford the product as a pale yellow waxy solid (91%). ¹H NMR (400 MHz, DMSO-d₆, 300 K) δ 1.10-1.38 (m, 6H), 1.47-1.56 (m, 1H), 1.64-1.99 (m, 10H), 2.05-2.14 (m, 2H), 2.31-2.49 (m, 5H, partially obscured by DMSO peak), 2.58-2.66 (m, 1H), 2.78-2.86 (m, 1H), 3.12-3.21 (m, 1H), 3.51-3.66 (m, 2H), 3.86 (s, 31H), 4.13-4.21 (m, 31H), 4.35-4.44 (m, 1H), 6.49 (d, J 16.2, 1H), 7.01-7.05 (m, 1H), 7.12 (br s, 1H), 7.26 (d, J 8.6, 1H), 7.56 (d, J 16.2, 1H), 7.60-7.68 (m, 5H), 7.80 (d, J 8.6, 1H), 8.06 (s, 1H), 8.29 (s, 1H), 9.67 (s, 1H); MS (ES⁺) m/z 703 (M+H)⁺

### Step 3: (2E)-3-(4-{[]-{[(14-cyclohexyl-3-methoxy-6-methyl-5,6,7,8-tetrahydroindolo[2,1-a][2,5]-benzodiazocin-11-yl)carbonyl]amino}cyclopentyl)carbonyl]amino}phenyl)acrylic Acid

Lithium hydroxide monohydrate (2.5 eq) was added to ethyl (2E)-3-(4-{[(1-{[(14-cyclohexyl-3-methoxy-6-methyl-5,6,7,8-tetrahydroindolo[2,1-a][2,5]benzodiazocin-11-yl)carbonyl]amino}cyclopentyl)carbonyl]amino}phenyl)acrylate (from Step 2) in a 1:1 methanol:water mixture (0.06 M). The reaction was stirred at 40° C. for 3 h, until complete hydrolysis of the ester had occurred. The reaction was allowed to cool to rt before being quenched with 1N HCl and the aqueous extracted (3 times) with EtOAc. The combined organics were washed with brine, dried over Na₂SO₄, filtered and concentrated in vacuo to afford the crude product as a brown solid. Purification was by automated RP-MS-HPLC (stationary phase: column WATERS XTERRA prep. C18, 5 um, 19×100 mm. Mobile phase: MeCN/H₂O buffered with 0.1% TFA). Fractions containing the pure compound were combined and freeze dried in the presence of 6N HCl to afford the HCl salt of the title compound as a white powder (33%). ¹H NMR (400 MHz, DMSO-d₆+TFA, 300 K) δ 1.10-1.20 (m, 1H), 1.25-1.38 (m, 2H), 1.49-1.58 (m, 1H), 1.64-1.99 (m, 10H), 2.04-2.15 (m, 2H), 2.31-2.40 (m, 2H), 2.58-2.65 (m, 1H), 3.04 (br s, 3H), 3.38-3.49 (m, 1H), 3.60-3.76 (m, 3H), 3.90 (s, 3H), 4.29-4.36 (m, 1H), 4.69-4.77 (m, 1H), 6.38 (d, J 16.0, 1H), 7.20-7.25 (m, 1H), 7.39 (d, J 8.8, 1H), 7.50 (d, J 16.0, 1H), 7.56-7.61 (m, 3H), 7.65 (d, J 9.0, 2H), 7.74-7.78 (m, 1H), 7.85 (d, J 8.6, 1H), 8.16 (s, 1H), 8.38 (s, if H), 9.71 (s, 1H), 10.0 (br s, 1H); MS (ES⁺) m/z 675 (M+H)⁺

### EXAMPLE 2

### (2E)-3-(4-{[(1-{[(14-cyclohexyl-6-methyl-5,6,7,8-tetrahydroindolo[2,1-a][2,5]benzodiazocin-11-yl)carbonyl]amino}cyclopentyl)carbonyl]amino}phenyl)acrylic Acid

### Step 1: Methyl 3-cyclohexyl-1-(1,3-dioxolan-2-ylmethyl)-2-(2-formylphenyl)-1H-indole-6-carboxylate

To a solution of methyl 2-bromo-3-cyclohexyl-1-(1,3-dioxolan-2-ylmethyl)-1H-indole-6-carboxylate from (Description 1, Step 1) in dioxane (0.15 M) were added Na₂CO₃ (4 eq., 2 M aqueous solution), 2-formylphenylboronic acid (1.5 eq.) and bis(triphenylphosphine)palladium(II) dichloride (0.2 eq.). The mixture was degassed before being heated at reflux for 45 min. RP-HPLC analysis of the reaction mixture showed starting material persisted. Heating at reflux was therefore resumed for a further 30 min. The reaction was allowed to cool to RT and partitioned between water and EtOAc. The aqueous fraction was extracted with EtOAc and the combined organics washed with brine before being dried over Na₂SO₄, filtered and concentrated in vacuo. The crude material was purified by flash chromatography (10-20% gradient EtOAc/PE) to afford the title compound as a yellow foam (85%). ¹H NMR (300 MHz, DMSO-d_{6, 300} K) [symbol] 1.02-1.23 (m, 3H), 1.59-1.81 (m, 7H), 2.30-2.37 (m, 1H), 3.37-3.45 (m, 1H), 3.52-3.64 (m, 3H), 3.89 (s, 3H), 3.95-4.00 (m, 1H), 4.26 (dd, J 15.3, 2.5, 1H), 4.91-4.93 (m, 1H), 7.51 (d, J 7.1, 1H), 7.69-7.77 (m, 2H), 7.82-7.87 (m, 2H), 8.03 (d, J 7.7, 1H), 8.22 (s, 1H), 9.67 (s, 1H); MS (ES⁺) m/z 448 (M+H)⁺

### Step 2: Methyl 3-cyclohexyl-1-(1,3-dioxolan-2-ylmethyl)-2-{2-[methylamino)methyl]phenyl}-1H-indole-6-carboxylate

To a solution of methyl 3-cyclohexyl-1-(1,3-dioxolan-2-ylmethyl)-2-(2-formylphenyl)-1H-indole-6-carboxylate (from Step 1) in THF (0.15 M), methylamine (10 eq., 2 M solution in THF) was added and the pH adjusted to pH 6 with acetic acid. The solution was stirred at RT for 1 h before being concentrated in vacuo. The residue was taken up in MeOH to give a 0.075 M solution. NaBH₃CN (1.5 eq.) was added and the mixture stirred at RT for 1 h. RP-HPLC analysis of the reaction mixture showed the complete conversion to the desired amine. The reaction was quenched with saturated aqueous NaHCO₃ and extracted (twice) with EtOAc. The combined organics were washed with brine before being dried over Na₂SO₄, filtered and concentrated in vacuo to give the title compound as a viscous oil (quantitative). ¹H NMR (300 MHz, DMSO-d_{6, 300} K) [symbol] 1.12-1.29 (m, 4H), 1.60-1.75 (m, 6H), 2.18 (s, 3H), 2.27-2.32 (m, 1H), 3.36 (br s, 2H, partially obscured by water peak), 3.61-3.81 (m, 5H), 3.87 (s, 3H), 4.14-4.21 (m, 1H), 4.87 (t, J 4.2, 1H), 7.27 (d, J 7.3, 1H), 7.37-7.42 (m, 1H), 7.50-7.55 (m, 1H), 7.65-7.68 (m, 2H), 7.82 (d, J 8.4, 1H), 8.18 (s, 1H); MS (ES⁺) m/z 463 (M+H)⁺

### Step 3: 14-cyclohexyl-6-methyl-5,6,7,8-tetrahydroindolo[2,1-a][2,5]-benzodiazocine-11-carboxylic Acid

Aqueous HCl (25 eq., 3 M) was added to a solution of methyl 3-cyclohexyl-1-(1,3-dioxolan-2-ylmethyl)-2-{2-[(methylamino)methyl]phenyl}-1H-indole-6-carboxylate (from Step 2) in THF (0.02 M), and the mixture heated at reflux for 21 h. ¹H NMR analysis of an aliquot from the reaction mixture confirmed the complete consumption of starting material. The volatiles were reduced in vacuo, and the residue partitioned between EtOAc and saturated aqueous NaOH (2 M) (ensuring that the aqueous phase was basic). The aqueous phase was extracted with EtOAc and the combined organics washed with brine, before being dried over Na₂SO₄, filtered and concentrated in vacuo. The residue was redissolved in MeOH (0.06 M) and acetic acid was added dropwise to the stirred solution at RT, to adjust the pH to pH 6. The mixture was stirred for 10 min prior to introducing NaCNBH₃ (1.5 eq.). RP-HPLC analysis of the reaction mixture after 18 h confirmed the complete conversion of the aminoaldehyde to the desired cyclic amine. The reaction was diluted with an equal volume of THF and NaOH (50 eq., 2 M aqueous solution) introduced. The reaction mixture was then heated at 90° C. for 10 h before being allowed to cool to RT. The THF/MeOH volume was reduced in vacuo and the residue acidified with aqueous HCl (1 M) before being extracted with EtOAc (4×). The combined organics were washed with brine, dried over Na₂SO₄, filtered and concentrated in vacuo to afford the hydrochloride salt of the product as a yellow solid. Purification was by automated RP-MS-HPLC (stationary phase: column WATERS XTERRA prep. C18, 5 um, 19×100 mm. Mobile phase: MeCN/H₂O buffered with 0.1% TFA). Fractions containing the pure compound were combined and freeze dried to afford the title compound as a white powder (40%). ¹H NMR (400 MHz, DMSO-d₆+TFA, 300 K) δ 1.12-1.15 (m, 1H), 1.31-1.37 (m, 2H), 1.53-1.56 (m, 1H), 1.67-1.75 (m, 2H), 1.82-1.85 (m, 1H), 1.93-1.98 (m, 3H), 2.63-2.67 (m, 1H), 3.04 (br s, 3H), 3.65-3.78 (m, 4H), 4.38-4.41 (m, 1H), 4.79-4.83 (m, 1H), 7.47-7.49 (m, 1H), 7.66-7.68 (m, 2H), 7.74 (dd, J 8.3, 1H), 7.92-7.95 (m, 2H), 8.21 (s, 1H); MS (ES⁺) m/z 389 (M+H)⁺

### Step 4: (2E)-3-(4-{[(1-{[(14-cyclohexyl-6-methyl-5,6,7,8-tetrahydroindolo[2,1-a][2,5]-benzodiazocin-11-yl)carbonyl]amino}cyclopentyl)carbonyl]amino}phenyl) Acrylic Acid

Performed (starting from 14-cyclohexyl-6-methyl-5,6,7,8-tetrahydroindolo[2,1-a][2,5]benzodiazocine-11-carboxylic acid from Step 3 and ethyl (2E)-3-(4-{[(1-aminocyclopentyl)carbonyl]amino}phenyl)acrylate from Example 1, Step 1) in directly analogous fashion to Example 1, Steps 2 and 3. ¹H NMR (400 MHz, DMSO-d₆+TFA, 300 K) δ 1.05-1.17 (m, 2H), 1.25-1.33 (m, 2H), 1.52-1.54 (m, 1H), 1.68-1.74 (m, 6H), 1.87-1.96 (m, 3H), 2.09-2.11 (m, 2H), 2.34-2.36 (m, 2H), 2.63-2.67 (m, 1H), 3.02 (br s, 3H), 3.40-3.51 (m, 1H), 3.62-3.74 (m, 3H), 4.35-4.38 (m, 1H), 4.71-4.75 (m, 1H), 6.33-6.37 (d, J 16.0, 1H), 7.45-7.47 (m, 2H), 7.51-7.56 (m, 3H), 7.63-7.65 (m, 4H), 7.75-7.77 (m, 1H), 7.85-7.88 (m, 1H), 7.92 (s, 1H), 8.15 (s, 1H), 8.38 (s, 1H), 9.69 (s, 1H), 9.98 (br s, 1H); MS (ES⁺) m/z 645 (M+H)⁺

### EXAMPLE 3

### (2E)-3-{4-[({1-[({13-cyclohexyl-5-[2-(dimethylamino)ethyl]-6,7-dihydro-5H-indolo[1,2-d][1,4]benzodiazepin-10-yl}carbonyl)amino]cyclopentyl}carbonyl)amino]phenyl}acrylic Acid

### Step 1: Methyl 2-bromo-1-(2-tert-butoxy-2-oxoethyl)-3-cyclohexyl-1H-indole-6-carboxylate

NaH (1.4 eq., 60% dispersion in mineral oil) was added to a solution of methyl 2-bromo-3-cyclohexyl-1H-indole-6-carboxylate in DMF (0.2 M) and the solution allowed to stir at RT for 1 h. Then tert-butyl bromoacetate (1.1 eq.) was added and the mixture stirred at RT for 40 min. RP-HPLC analysis showed the absence of the starting material with the formation of a single main product. DMF was concentrated in vacuo and the residue taken up in EtOAc. The organic phase was washed with H₂O (twice) and then brine before being dried over Na₂SO₄, filtered and the solvent evaporated in vacuo. The title compound was obtained as a brownish solid (90%). ¹H NMR (400 MHz, DMSO-d₆, 300 K) δ 1.3-1.50 (m, 12H), 1.65-1.80 (m, 3H), 1.80-2.00 (m, 4H), 2.85-2.95 (m, 1H), 3.87 (s, 3H), 5.1 (s, 2H), 7.67 (d, J 8.4, 1H), 7.83 (d, J 8.4, 1H), 8.13 (s, 1H); MS (ES⁺) m/z 450 (M+H)⁺, m/z 452 (M+H)⁺

### Step 2: Methyl 2-{2-[(tert-butoxycarbonyl)amino]phenyl}-1-(2-tert-butoxy-2-oxoethyl)-3-cyclohexyl-1H-indole-6-carboxylate

To a solution of methyl 2-bromo-1-(2-tert-butoxy-2-oxoethyl)-3-cyclohexyl-1H-indole-6-carboxylate (from Step 1) in dioxane (0.15 M) was added Na₂CO₃ (4 eq., 2 M aqueous solution), tert-butyl [2-(4,4,5,5-tetramethyl-1,3,2-dioxaborolan-2-yl)phenyl]carbamate (1.5 eq.) and bis(triphenylphosphine)palladium(II) dichloride (0.2 eq.). The mixture was heated at reflux for 1 h, at which point RP-HPLC analysis showed the complete consumption of the starting material. The reaction mixture was filtered and then the filtrate was diluted with EtOAc. The organic phase was washed with H₂O, brine and dried over Na₂SO₄ before being filtered and concentrated in vacuo. The crude was purified by flash chromatography (BIOTAGE cartridge Si40M, 0.8:9.2 EtOAc/PE) to afford the title compound as a white solid (65%). ¹H NMR (400 MHz, DMSO-d₆, 300 K) [symbol] 1.10-1.40 (m, 20H), 1.50-1.90 (m, 8H), 2.20-2.40 (m, 1H), 3.87 (s, 3H), 4.51-4.73 (m, 2H), 7.10-7.22 (m, 2H), 7.45-7.52 (m, 1H), 7.65-7.71 (m, 2H), 7.78-7.82 (m, if H), 7.86 (d, J 8.4, 1H), 8.10 (s, 1H); MS (ES⁺) m/z 563 (M+H)⁺

### Step 3: Methyl 13-cyclohexyl-6-oxo-6,7-dihydro-5H-indolo[1,2-d][1,4]-benzodiazepine-10-carboxylate

To a solution of methyl 2-{2-[(tert-butoxycarbonyl)amino]phenyl}-1-(2-tert-butoxy-2-oxoethyl)-3-cyclohexyl-1H-indole-6-carboxylate (from Step 2) in a 1:1 mixture of DCM/H₂O (0.06 M) a large excess (>200 eq.) of TFA was added and the solution was heated for 10 h at 40° C. RP-HPLC analysis showed complete consumption of the starting material. The solvents were removed in vacuo. The crude residue was purified by flash chromatography (BIOTAGE cartridge Si40M, 4:6 EtOAc/PE) to afford the title compound as a white solid (74%). ¹H NMR (400 MHz, DMSO-d₆, 300 K) δ 1.10-1.20 (m, 1H), 1.30-1.55 (m, 3H), 1.65-1.80 (m, 2H), 1.81-1.95 (m, 1H), 1.98-2.11 (m, 3H) 2.80-2.92 (m, 1H), 3.90 (s, 3H), 4.54 (d, J 14.7, 1H), 5.08 (d, J 14.7, 1H), 7.29 (d, J 7.8, 1H), 7.38-7.42 (m, 1H), 7.51-7.55 (m, 2H), 7.69 (d, J 8.5, 1H), 7.97 (m, J 8.5 1H), 8.28 (s, 1H), 10.3 (s, 1H); MS (ES⁺) m/z 389 (M+H)⁺

### Step 4: Methyl 13-cyclohexyl-5-[2-(dimethylamino)-2-oxoethyl]-6-oxo-6,7-dihydro-5H-indolo[1,2-d][1,4]-benzodiazepine-10-carboxylate

NaH (1.4 eq., 60% dispersion in mineral oil) was added to a solution of methyl 13-cyclohexyl-6-oxo-6,7-dihydro-5H-indolo[1,2-d][1,4]benzodiazepine-10-carboxylate (from Step 3) in DMF (0.15 M) and the solution was allowed to stir at RT for 45 min. Then 2-chloro-N,N-dimethylacetamide (1.1 eq.) was added and the mixture stirred at RT for 20 min. RP-HPLC analysis showed the absence of starting material with desired product as the main peak. The solution was diluted with EtOAc and washed with 1N HCl. The aqueous phase was extracted with EtOAc (×3) and the combined organics washed with brine, dried over Na₂SO₄, filtered and the solvent evaporated in vacuo. The crude product was purified by flash chromatography (BIOTAGE cartridge Si12M, 9:1 EtOAc/PE) to give the title compound (73%). ¹H NMR (400 MHz, DMSO-d₆, 300 K) δ 1.15-1.23 (m, 1H), 1.35-1.48 (m, 2H), 1.55-1.61 (m, 1H) 1.70-1.77—(m, 2H), 1.85-2.11, (m, 4H), 2.46-2.51 (m, 1H partially obscured by DMSO peak), 2.82 (s, 3H), 2.90 (s, 3H), 3.89 (s, 3H), 4.42 (d, J 16.8, 1H), 4.49 (d, J 14.6, 1H), 4.66 (d, J 16.8, 1H), 5.20 (d, J 14.6, 1H), 7.42-7.49 (m, 2H), 7.52-7.68 (m, 2H), 7.69 (d, J 8.8, 1H), 7.96 (d, J 8.8, 1H), 8.29 (s, 1H); MS (ES⁺) m/z 474(M+H)⁺

### Step 5: Methyl 13-cyclohexyl-5-[2-(dimethylamino)ethyl]-6,7-dihydro-5H-indolo[1,2-d][1,4]-benzodiazepine-10-carboxylate

To a solution of methyl 13-cyclohexyl-5-[2-(dimethylamino)-2-oxoethyl]-6-oxo-6,7-dihydro-5H-indolo[1,2-d][1,4]benzodiazepine-10-carboxylate (from Step 4) in THF (0.15 M), BH₃.Me₂S (20 eq., 2 M sol. in THF) was added and the mixture was stirred at RT for 3 h. RP-HPLC analysis confirmed the absence of starting material with the formation of a new single main product. The solution was carefully quenched by adding 1.25 N HCl in MeOH until effervescence subsided. Then the volatiles were driven off by boiling the mixture to dryness. The crude residue was used directly in the next step. MS (ES⁺) m/z 446 (M+H)⁺

### Step 6: 13-cyclohexyl-5-[2-(dimethylamino)ethyl]-6,7-dihydro-5H-indolo[1,2-d][1,4]benzodiazepine-10-carboxylic Acid

To a solution of methyl 13-cyclohexyl-5-[2-(dimethylamino)ethyl]-6,7-dihydro-5H-indolo[1,2-d][1,4]benzodiazepine-10-carboxylate (from Step 5) in MeOH (0.06 M), 5 eq 1N NaOH was added. The solution was stirred at 60° C. for 5 h, at which point RP-HPLC analysis showed the absence of starting material and the formation of the product as the major peak. The solvent was evaporated in vacuo. The crude was then purified by prep RP-HPLC (stationary phase: column WATERS XTERRA prep. C18, 5 um, 19×150 mm. Mobile phase: MeCN/H₂O buffered with 0.1% TFA). Fractions containing the pure compound were combined and freeze dried to afford the title compound as a yellow powder in a 35% yield (over two steps). ¹H NMR (400 MHz, DMSO-d₆, 300 K) δ 1.15-1.55 (m, 4H), 1.70-2.10 (m, 6H), 2.46 (s, 6H), 2.70-2.82 (m, 1H), 3.00-3.20 (m, 3H), 3.50-4.00 (m, 4H), 4.70-4.85 (br s, 1H), 7.25-7.38 (m, 3H), 7.50 (t, J 7.2, 1H), 7.61 (d, J 8.4, 1H), 7.86 (d, J 8.4, 1H), 8.12 (s, 1H), 9.14 (br s, 1H), 12.6 (br s, 1H); MS (ES⁺) m/z 432 (M+H)⁺

### Step 7: (2E)-3-{4-[({1-[({13-cyclohexyl-5-[2-(dimethylamino)ethyl]-6,7-dihydro-5H-indolo[1,2-d][1,4]-benzodiazepin-10-yl}carbonyl)amino]cyclopentyl}carbonyl)amino]phenyl}acrylic Acid

Performed (starting from 13-cyclohexyl-5-[2-(dimethylamino)ethyl]-6,7-dihydro-5H-indolo[1,2-d][1,4]benzodiazepine-10-carboxylic acid from Step 6 and ethyl (2E)-3-(4-{[(1-aminocyclopentyl)carbonyl]amino}phenyl)acrylate from Example 1, Step 1) in directly analogous fashion to Example 1, Steps 2 and 3—omitting the acidic wash in both aqueous work-ups. ¹H NMR (400 MHz, DMSO-d₆, 300 K) δ 1.15-1.46 (m, 4H), 1.60-1.90 (m, 8H), 1.95-2.20 (m, 4H), 2.30-2.40 (m, 2H), 2.46 (s, 6H), 2.75-2.85 (m, 1H), 3.00-4.00 (m, 3H), 3.50-4.00 (m, 5H partially obscured by water peak), 6.38 (d, J 16.0, 1H), 7.27-7.40 (m, 3H), 7.50 (d, J 16, 1H), 7.48-7.52 (m, 1H), 7.54-7.70 (m, 6H), 7.81 (d, J 8.5, 1H), 8.18 (s, 1H), 8.29 (s, 1H), 9.14 (br s, 1H), 9.67 (s, 1H), 12.20 (br s, 1H); MS (ES⁺) m/z 688 (M+H)⁺

### EXAMPLE 4

### (2E)-3-{4-[({1-[({14-cyclohexyl-6-[2-(dimethylamino)ethyl]-5,6,7,8-tetrahydroindolo[2,1-a][2,5]benzodiazocin-11-yl}carbonyl)amino]cyclopentyl}carbonyl)amino]phenyl}acrylic Acid

Performed (starting from 14-cyclohexyl-6-[2-(dimethylamino)ethyl]-5,6,7,8-tetrahydroindolo[2,1-a][2,5]benzodiazocine-11-carboxylic acid-(from Description 2)- and ethyl (2E)-3-(4-{[(1-aminocyclopentyl)carbonyl]amino}phenyl)acrylate from Example 1, Step 1) in directly analogous fashion to Example 1, Steps 2 and 3—omitting the acidic wash in both aqueous work-ups. ¹H NMR (400 MHz, DMSO-d₆+TFA, 300K) δ 1.10-1.20 (m, 1H), 1.25-1.40 (m, 2H), 1.50-1.60 (m, 1H), 1.65-1.85 (m, 7H), 1.90-2.00 (m, 3H), 2.11-2.16 (m, 2H), 2.29-2.43 (m, 2H), 2.50-2.70 (m, 1H), 2.85 (s, 6H), 3.38-3.50 (m, 1H), 3.50-3.77 (m, 7H), 4.40 (d, J 13.6, 1H), 4.77 (m, 1H), 6.56 (d, J 16.0, 1H), 7.46-7.52 (m, 2H), 7.55-7.60 (m, 2H), 7.62-7.69 (m, 4H), 7.80-7.91 (m, 3H), 8.11 (s, 1H), 8.43 (s, 1H), 9.71 (s, 1H); MS (ES⁺) m/z 702 (M+H)⁺

### EXAMPLE 5

### (2E)-3-{4-[({1-[({(7R)-14-cyclohexyl-7-[[2-(dimethylamino)ethyl](methyl)amino]-7,8-dihydro-6H-indolo[1,2-e][1,5]benzoxazocin-11-yl}carbonyl)amino]cyclopentyl}carbonyl)amino]phenyl}acrylic Acid

To a solution of (7R)—N-(11-carboxy-14-cyclohexyl-7,8-dihydro-6H-indolo[1,2-e][1,5]benzoxazocin-7-yl)-N,N′,N′-trimethylethane-1,2-diaminium bis(trifluoroacetate) (prepared as described in International Patent Application publication WO 2006/04603, Examples 9 and 12) and the hydrochloride salt of methyl (2E)-3-(4-{[(1-aminocyclopentyl)carbonyl]-amino}phenyl)acrylate (1.1. eq, prepared as outlined in Example 1, Step 1 for the ethyl ester) in dry DMF (0.14M) were added DIPEA (6 eq.) and HATU (1.5 eq.) and the resulting mixture was left stirring at RT for 60 h. The mixture was then diluted with EtOAc and extracted with saturated aqueous NaHCO₃-solution and with brine. After drying over Na₂SO₄ all volatiles were evaporated in vacuo and the residual material was dissolved in THF/MeOH/water (4:1:1) and lithium hydroxide monohydrate (1.5 eq.) was added. The mixture was left stirring for 3 h at 40° C. All volatiles were then evaporated in vacuo and the residual material was purified by mass-guided prep. RP-HPLC (stationary phase: column WATERS XTERRA prep. C18, 5 um, 19×150 mm. Mobile phase: MeCN/H₂O buffered with 0.1% TFA). After lyophilization of the product fractions the product was obtained as a colorless powder. ¹H NMR (400 MHz, DMSO-d_{6, 300} K) δ 1.15-1.56 (m, 5H), 1.70-1.99 (m, OH), 2.07-2.14 (m, 2H), 2.29-2.38 (m, 5H), 2.65-2.74 (m, 6H), 2.82-2.92 (m, 1H), 3.03-3.26 (m, 4H), 3.39 (d, J 7.13, 1H), 3.78-3.84 (m, 1H), 4.02-4.08 (m, 1H); 4.26-4.30 (m, 1H), 4.60-4.64 (m, 1H), 6.39 (d, J 15.9, 1H), 7.26-7.33 (m, 3H), 7.48-7.66 (m, 6H), 7.75-7.77 (m, 1H), 7.80-7.83 (m, 1H), 8.06 (s, 1H), 8.36 (s, 1H), 9.68 (s, 1H); MS (ES⁺) m/z 732.8 (M+H)⁺.

1. A compound of the formula (I): 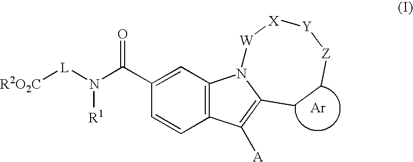
wherein
Ar is a moiety containing at least one aromatic ring and possesses 5-, 6-, 9- or 10-ring atoms optionally containing 1, 2 or 3 heteroatoms independently selected from N, O and S, which ring is optionally substituted by groups Q¹ and Q²;
Q¹ is halogen, hydroxy, C₁₋₆alkyl, C₁₋₆alkoxy, aryl, heteroaryl, CONR^{a}R^{b},
(CH₂)₀₋₃NR^{a}R^{b}, O(CH₂)₁₋₃NR^{a}R^{b}, O(CH₂)₀₋₃CONR^{a}R^{b}, O(CH₂)₀₋₃aryl, O(CH₂)₀₋₃heteroaryl, OCHR^{c}R^{d};
R^{a} and R^{b} are each independently selected from hydrogen, C₁₋₄alkyl and C(O)C₁₋₄alkyl;
or R^{a}, R^{b} and the nitrogen atom to which they are attached form a heteroaliphatic ring of 4 to 7 ring atoms, where said ring is optionally substituted by halogen, hydroxy, C₁₋₄alkyl or C₁₋₄alkoxy;
R^{c} and R^{d} are each independently selected from hydrogen and C₁₋₄alkoxy;
or R^{c} and R^{d} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;
and wherein said C₁₋₄alkyl, C₁₋₄alkoxy and aryl groups are optionally substituted by halogen or hydroxy;
Q² is halogen, hydroxy, C₁₋₄alkyl or C₁₋₄alkoxy, where said C₁₋₄alkyl and C₁₋₄alkoxy groups are optionally substituted by halogen or hydroxy;
or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄alkoxy;
A is C₃₋₆alkyl or C₂₋₆alkenyl,
or A is a non-aromatic ring of 3 to 8 ring atoms where said ring may contain a double bond and/or may contain a O, S, SO, SO₂ or NH moiety,
or A is a non-aromatic bicyclic moiety of 4 to 8 ring atoms,
and A is optionally substituted by halogen, hydroxy, C₁₋₄alkyl or C₁₋₄alkoxy;
R¹ is hydrogen, C₁₋₆alkyl or C₂₋₆alkenyl;
R² is hydrogen or C₁₋₆alkyl;
L is 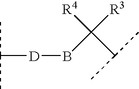
wherein R³ and R⁴ are each independently selected from hydrogen, halogen, C₁₋₄alkyl, C₂₋₄alkenyl or C₁₋₄alkoxy;
or R³ and R⁴ are linked to form a C₃₋₈cycloalkyl group;
B is aryl, heteroaryl, CONR⁵R⁶, optionally substituted by halogen, C₁₋₄alkyl, C₂₋₄alkenyl or C₁₋₄alkoxy;
R⁵ is hydrogen or C₁₋₆alkyl;
or R⁵ is linked to R³ and/or R⁴ to form a 5- to 10-membered ring, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;
R⁶ is aryl or heteroaryl;
or R⁵, R⁶ and the nitrogen atom to which they are attached form a 5- to 10-membered mono- or bi-cyclic ring system, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;
D is a bond, C₁₋₆alkylene, C₂₋₆alkenylene, C₂₋₆alkynylene, aryl or heteroaryl, where said aryl or heteroaryl is optionally substituted by halogen, C₁₋₄alkyl or C₂₋₄alkenyl;
W and Z are independently selected from a bond, C═O, O, S(O)₀₋₂, —(CR¹⁰R¹¹)—(CR¹²R¹³)₀₋₁— and NR¹⁰;
X and Y are independently selected from a bond, C═O, O, —CR¹⁴R¹⁵— and NR¹⁴;
and none, one or two of W, X, Y and Z are a bond;
R¹⁰, R¹¹, R¹², R¹³, R¹⁴ and R¹⁵ are each independently selected from hydrogen, hydroxy, C₁₋₆alkyl, C₂₋₆alkenyl, C₁₋₆alkoxy, C(O)C₁₋₆alkyl, Het, (CH₂)₀₋₃NR¹⁶R¹⁷, C(O)(CH₂)₀₋₃NR¹⁶R¹⁷, NHC(O)(CH₂)₀₋₃NR¹⁶R¹⁷, O(CH₂)₁₋₃NR¹⁶R¹⁷, S(O)₀₋₂(CH₂)₀₋₃R¹⁶R¹⁷ and C(O)(CH₂)₀₋₃OR¹⁶;
Het is a heteroaliphatic ring of 4 to 7 ring atoms, which ring may contain 1, 2 or 3 heteroatoms selected from N, O or S or a group S(O), S(O)₂, NH or NC₁₋₄alkyl;
R¹⁶ and R¹⁷ are independently selected from hydrogen, C₁₋₆alkyl and (CH₂)₀₋₄NR¹⁸R¹⁹;
or R⁶, R¹⁷ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;
R¹⁸ and R¹⁹ are independently selected from hydrogen and C₁₋₆alkyl; or R¹⁸, R¹⁹ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;
and pharmaceutically acceptable salts thereof.

2. The compound as claimed in claim 1, wherein Ar is a 5- or 6-membered aromatic ring optionally containing 1, 2 or 3 heteroatoms independently selected from N, O and S, and which ring is optionally substituted by groups Q¹ and Q² as defined in claim 1.

3. The compound as claimed in claim 1, wherein A is C₃₋₆alkyl, C₂₋₆alkenyl or C₃₋₈cycloalkyl, where A is optionally substituted by halogen, hydroxy, C₁₋₄alkyl or C₁₋₄alkoxy.

4. The compound as claimed in claim 1, wherein R¹ is hydrogen or C₁₋₄alkyl.

5. The compound as claimed in claim 1 wherein R² is hydrogen or C₁₋₄alkyl.

6. The compound as claimed in claim 1, wherein R³ and R⁴ are linked to form a cyclobutyl, cyclopentyl or cyclohexyl group.

7. The compound as claimed in claim 1, wherein B is CONR⁵aryl, optionally substituted by halogen, C₁₋₄alkoxy, where R⁵ is as defined in claim 1.

8. The compound as claimed in claim 1, wherein D is a bond or ethenylene.

9. The compound as claimed in claim 1, wherein W is a bond, C═O, —CR¹⁰R¹¹)—(CR¹²R¹³)₀₋₁— or NR¹⁰ where R¹⁰, R¹¹, R¹² and R¹³ are as defined in claim 1.

10. The compound as claimed in claim 1, wherein Z is a bond, C═O, O, —CR¹⁰R¹¹)—(CR¹²R¹³)₀₋₁— or NR¹⁰ where R¹⁰, R¹¹, R¹² and R¹³ are as defined in claim 1.

11. The compound as claimed in claim 1, wherein X is C═O, —CR¹⁴R¹⁵— or NR¹⁴ where R¹⁴ and R¹⁵ are as defined in claim 1.

12. The compound as claimed in claim 1, wherein Y is C═O, O, —CR¹⁴R¹⁵— or NR¹⁴ where R¹⁴ and R¹⁵ are as defined in claim 1.

13. The compound as claimed in claim 1 of formula (Ia): 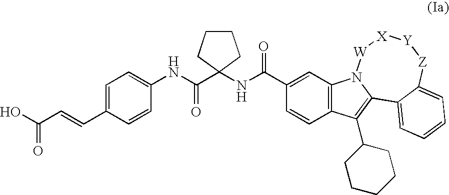
or a pharmaceutically acceptable salt thereof, wherein W, X, Y and Z are as defined in claim 1.

14. The compound as claimed in claim 1 selected from:
(2E)-3-(4-{[(1-{[(14-cyclohexyl-3-methoxy-6-methyl-5,6,7,8-tetrahydroindolo[2,1-a][2,5]benzodiazocin-11-yl)carbonyl]amino}cyclopentyl)carbonyl]amino}phenyl)acrylic acid,
(2E)-3-(4-{[(1-{[(14-cyclohexyl-6-methyl-5,6,7,8-tetrahydroindolo[2,1-a][2,5]benzodiazocin-11-yl)carbonyl]amino}cyclopentyl)carbonyl]amino}phenyl)acrylic acid,
(2E)-3-{4-[({1-[({13-cyclohexyl-5-[2-(dimethylamino)ethyl]-6,7-dihydro-5H-indolo[1,2-d][1,4]benzodiazepin-10-yl}carbonyl)amino]cyclopentyl}carbonyl)amino]phenyl}acrylic acid,
(2E)-3-{4-[({1-[({14-cyclohexyl-6-[2-(dimethylamino)ethyl]-5,6,7,8-tetrahydroindolo[2,1-a][2,5]benzodiazocin-11-yl}carbonyl)amino]cyclopentyl}carbonyl)amino]phenyl}acrylic acid,
(2E)-3-{4-[({1-[({(7R)-[4-cyclohexyl-7-[[2-(dimethylamino)ethyl](methyl)amino]-7,8-dihydro-6H-indolo[1,2-e][1,5]benzoxazocin-11-yl}carbonyl)amino]cyclopentyl}carbonyl)amino]phenyl}acrylic acid,
and pharmaceutically acceptable salts thereof.

15. The compound as claimed in claim 1 or a pharmaceutically acceptable salt thereof for use in therapy.

16. (canceled)

17. A pharmaceutical composition comprising a compound as claimed in claim 1, or a pharmaceutically acceptable salt thereof, in association with a pharmaceutically acceptable carrier.

18. The pharmaceutical composition as claimed in claim 17, further comprising one or more other agents for the treatment of viral infections such as an antiviral agent, or an immunomodulatory agent such as α-, β- or γ-interferon.

19. A method of inhibiting hepatitis C virus polymerase and/or of treating or preventing an illness due to hepatitis C virus, the method comprising administering to a human or animal (preferably mammalian) subject suffering from the condition a therapeutically or prophylactically effective amount of a a compound of the formula (I): 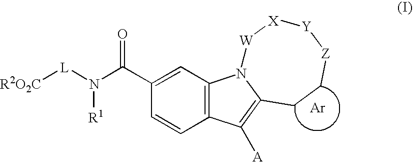
wherein
Ar is a moiety containing at least one aromatic ring and possesses 5-, 6-, 9- or 10-ring atoms optionally containing 1, 2 or 3 heteroatoms independently selected from N, O and S, which ring is optionally substituted by groups Q¹ and Q²;
Q¹ is halogen, hydroxy C₁₋₆alkyl, C₁₋₆alkoxy, aryl, heteroaryl, CONR^{a}R^{b}, (CH₂)₀₋₃NR^{a}R^{b}, O(CH₁₋₃NR^{a}R^{b}, O(CH₂)₀₋₃CONR^{a}R^{b}, O(CH₂)₀₋₃aryl, O(CH₂)₀₋₃heteroaryl, OCHR^{c}R^{d};
R^{a} and R^{b} are each independently selected from hydrogen, C₁₋₄alkyl and C(O)C₁₋₄alkyl;
or R^{a}, R^{b} and the nitrogen atom to which they are attached form a heteroaliphatic ring of 4 to 7 ring atoms, where said ring is optionally substituted by halogen, hydroxy, C₁₋₄alkyl or C₁₋₄alkoxy;
R^{c} and R^{d} are each independently selected from hydrogen and C₁₋₄alkoxy;
or R^{c} and R^{d} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;
and wherein said C₁₋₄alkyl, C₁₋₄alkoxy and aryl groups are optionally substituted by halogen or hydroxy;
Q² is halogen, hydroxy, C₁₋₄alkyl or C₁₋₄alkoxy, where said C₁₋₄alkyl and C₁₋₄alkoxy groups are optionally substituted by halogen or hydroxy;
or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄ alkoxy;
A is C₃₋₆alkyl or C₂₋₆alkenyl,
or A is a non-aromatic ring of 3 to 8 ring atoms where said ring may contain a double bond and/or may contain a O, S, SO, SO₂ or NH moiety,
or A is a non-aromatic bicyclic moiety of 4 to 8 ring atoms,
and A is optionally substituted by halogen, hydroxy, C₁₋₄alkyl or C₁₋₄alkoxy;
R¹ is hydrogen, C₁₋₆alkyl or C₂₋₆alkenyl;
R² is hydrogen or C₆ alkyl;
L is 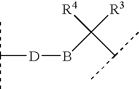
wherein R³ and R⁴ are each independently selected from hydrogen, halogen, C₁₋₄alkyl, C₂₋₄alkenyl or C₁₋₄alkoxy;
or R³ and R⁴ are linked to form a C₃₋₈cycloalkyl group;
B is aryl, heteroaryl, CONR⁵R⁶, optionally substituted by halogen, C₁₋₄alkyl, C₂₋₄alkenyl or C₁₋₄alkoxy;
R⁵ is hydrogen or C₁₋₆alkyl;
or R⁵ is linked to R³ and/or R⁴ to form a 5- to 10-membered ring, where said ring may be saturated, partially saturated or unsaturated, and where said ring is optionally substituted by halogen, C₁alkyl, C₄alkenyl, C₄alkynyl or C₁₋₄alkoxy;
R⁶ is aryl or heteroaryl;
or R⁵, R⁶ and the nitrogen atom to which they are attached form a 5- to 10-membered mono- or bi-cyclic ring system, where said ring may be saturated, partially saturated or unsaturated and where said ring is optionally substituted by halogen, C₁₋₄alkyl, C₁₋₄alkenyl, C₂₋₄alkynyl or C₁₋₄alkoxy;
D is a bond, C₁₋₆alkylene, C₂₋₆alkenylene, C₂₋₆alkynylene, aryl or heteroaryl, where said aryl or heteroaryl is optionally substituted by halogen, C₁₋₄alkyl or C₁₋₄alkenyl;
W and Z are independently selected from a bond, C═O, O, S(O)₀₋₂, —(CR¹⁰R¹¹)—(CR¹²R¹³)₀₋₁— and NR¹⁰;
X and Y are independently selected from a bond, C═O, O—CR¹⁴R¹⁵— and NR¹⁴;
and none, one or two of W, X, Y and Z are a bond;
R¹⁰, R¹¹, R¹², R¹³, R¹⁴ and R¹⁵ are each independently selected from hydrogen, hydroxy, C₁₋₆alkyl, C₁₋₆alkenyl C₁₋₆alkoxy, C(O)C₁₋₆alkyl, Het, (CH₂)₀₋₃NR¹⁶R¹⁷, C(O)(CH₂)₀₋₃NR¹⁶R¹⁷, NHC(O)(CH₂)₀₋₃NR¹⁶R¹⁷, O(CH₂)₁₋₃NR¹⁶R¹⁷, S(O)₀₋₂(CH₂)₀₋₃R¹⁶R¹⁷ and C(O)(CH₂)₀₋₃OR¹⁶;
Het is a heteroaliphatic ring of 4 to 7 ring atoms, which ring may contain 1, 2 or 3 heteroatoms selected from N, O or S or a group S(O), S(O)₂, NH or NC₁₋₄alkyl;
R¹⁶ and R¹⁷ are independently selected from hydrogen, C₁₋₆alkyl and (CH₂)₀₋₄NR¹⁸R¹⁹;
or R¹⁶, R¹⁷ and the nitrogen atom to which they are attached form a heteroaliphatic ring of 4 to 7 ring atoms, which ring may optionally contain 1 or 2 more heteroatoms selected from O or S or a group S(O), S(O)₉, NH or NC₁₋₄alkyl and which ring is optionally substituted by halogen, hydroxy, C₁₋₄alkyl or C₁₋₄alkoxy;
R¹⁸ and R¹⁹ are independently selected from hydrogen and C₁₋₆alkyl; or R¹⁸, R¹⁹ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl, and which ring is optionally substituted by halogen, hydroxy, C₁₋₄alkyl or C₁₋₄alkoxy;
and pharmaceutically acceptable salts thereof.

20. A process for the preparation of a compound of formula (I) by the reaction of a compound of formula (II) with a compound of formula (III): 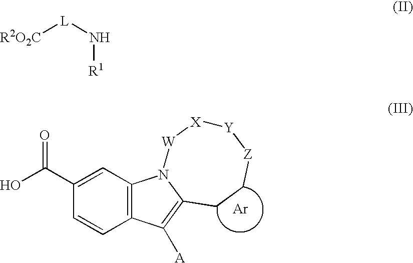
where R¹, R², L, A, Ar, W, X, Y and Z are as defined below, in the presence of a coupling reagent and a base in a suitable solvent, wherein the a compound of the formula (I): 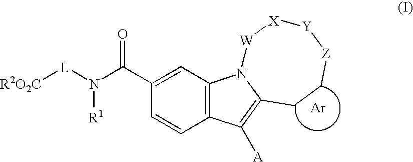
wherein
Ar is a moiety containing at least one aromatic ring and possesses 5-, 6-, 9- or 10-ring atoms optionally containing 1, 2 or 3 heteroatoms independently selected from N, O and S, which ring is optionally substituted by groups Q¹ and Q²;
Q¹ is halogen hydroxy C₁₋₆alkyl C₁₋₆alkoxy aryl, heteroaryl CONR^{a}R^{b}, (CH₂)₀₋₃NR^{a}R^{b}, O(CH₂)₁₋₃NR^{a}R^{b}, O(CH₂)₀₋₃CONR^{a}R^{b}, O(CH₂)₀₋₃aryl, O(CH₂)₀₋₃heteroaryl, OCHR^{c}R^{d};
R^{a} and R^{b} are each independently selected from hydrogen, C₁₋₄alkyl and C(O)C₁₋₄alkyl;
or R^{a}, R^{b} and the nitrogen atom to which they are attached form a heteroaliphatic ring of 4 to 7 ring atoms, where said ring is optionally substituted by halogen, hydroxy, C₁₋₄alkyl or C₁₋₄alkoxy;
R^{c} and R^{d} are each independently selected from hydrogen and C₁₋₄alkoxy;
or R^{c} and R^{d} are linked by a heteroatom selected from N, O and S to form a heteroaliphatic ring of 4 to 7 ring atoms, where said ring is optionally substituted by halogen, hydroxy, C₁₋₄alkyl or C₁₋₄alkoxy;
and wherein said C₁₋₄alkyl, C₁₋₄alkoxy and aryl groups are optionally substituted by halogen or hydroxy;
Q² is halogen, hydroxy, C₁₋₄alkyl or C₁₋₄alkoxy, where said C₁₋₄alkyl and C₁₋₄alkoxy groups are optionally substituted by halogen or hydroxy;
or Q¹ and Q² may be linked by a bond or a heteroatom selected from N, O and S to form a ring of 4 to 7 atoms, where said ring is optionally substituted by halogen, hydroxy, C₁₋₄alkyl or C₁₋₄alkoxy;
A is C₃₋₆alkyl or C₂₋₆alkenyl,
or A is a non-aromatic ring of 3 to 8 ring atoms where said ring may contain a double bond and/or may contain a O, S, SO, SO₂ or NH moiety,
or A is a non-aromatic bicyclic moiety of 4 to 8 ring atoms,
and A is optionally substituted by halogen, hydroxy, C₁₋₄alkyl or C₁₋₄alkoxy;
R¹ is hydrogen C₁₋₆alkyl or C₂₋₆alkenyl;
R² is hydrogen or C₁₋₆alkyl;
L is 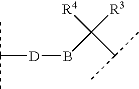
wherein R³ and R⁴ are each independently selected from hydrogen halogen, C₁₋₄alkyl, C₂₋₄alkenyl or C₁₋₄alkoxy;
or R³ and R⁴ are linked to form a C₃₋₈cycloalkyl group;
B is aryl, heteroaryl, CONR⁵R⁶, optionally substituted by halogen. C₁₋₄alkyl, C₂₋₄alkenyl or C₁₋₄alkoxy;
R⁵ is hydrogen or C₁₋₆alkyl;
or R⁵ is linked to R³ and/or R⁴ to form a 5- to 10-membered ring, where said ring may be saturated, partially saturated or unsaturated, and where said ring is optionally substituted by halogen, C₁₋₄alkyl, C₂₋₄alkenyl, C₂₋₄alkynyl or C₁₋₄alkoxy;
R⁶ is aryl or heteroaryl;
or R⁵, R⁶ and the nitrogen atom to which they are attached form a 5- to 10-membered mono- or bi-cyclic ring system where said ring may be saturated, partially saturated or unsaturated and where said ring is optionally substituted by halogen, C₁₋₄alkyl, C₂₋₄alkenyl, C₂₋₄alkynyl or C₁₋₄alkoxy;
D is a bond, C₁₋₆alkylene C₂₋₆alkenylene, C₂₋₆alkynylene, aryl or heteroaryl, where said aryl or heteroaryl is optionally substituted by halogen, C₁₋₄alkyl or C₁₋₄alkenyl;
W and Z are independently selected from a bond, C═O, O, S(O)₀₋₂, —(CR¹⁰R¹¹)—(CR¹²R¹³)₀₋₁— and NR¹⁰;
X and Y are independently selected from a bond, C═O, O—CR¹⁴R¹⁵— and NR¹⁴;
and none, one or two of W, X, Y and Z are a bond;
R¹⁰, R¹¹, R¹², R¹³, R¹⁴ and R¹⁵ are each independently selected from hydrogen, hydroxy, C₁₋₆alkyl, C₂₋₆-alkenyl C₁₋₆alkoxy, C(O)C₁₋₆alkyl Het, (CH₂)₀₋₃NR¹⁶R¹⁷, C(O)(CH₂)₀₋₃NR¹⁶R¹⁷, NHC(O)(CH₂)₀₋₃NR¹⁶R¹⁷, O(CH₂)₁₋₃NR¹⁶R¹⁷, S(O)₀₋₂(CH₂)₀₋₃R¹⁶R¹⁷ and C(O)(CH₂)₀₋₃OR¹⁶;
Het is a heteroaliphatic ring of 4 to 7 ring atoms which ring may contain 1, 2 or 3 heteroatoms selected from N, O or S or a group S(O), S(O)₂, NH or NC₁₋₄alkyl;
R¹⁶ and R¹⁷ are independently selected from hydrogen C₁₋₆alkyl and (CH₂)₀₋₄NR¹⁸R¹⁹;
or R¹⁶, R¹⁷ and the nitrogen atom to which they are attached form a heteroaliphatic ring of 4 to 7 ring atoms, which ring may optionally contain 1 or 2 more heteroatoms selected from O or S or a group S(O), S(O)₂, NH or NC₁₋₄alkyl and which ring is optionally substituted by halogen hydroxy C₁₋₄alkyl or C₁₋₄alkoxy;
R¹⁸ and R¹⁹ are independently selected from hydrogen and C₁₋₆ alkyl;
or R¹⁸, R¹⁹ and the nitrogen atom to which they are attached form a heteroaliphatic rind of 4 to 7 ring atoms, which ring may optionally contain 1 or 2 more heteroatoms selected from O or S or a group S(O), S(O)₂, NH or NC₁₋₄alkyl and which ring is optionally substituted by halogen, hydroxy, C₁₋₄alkyl or C₁₋₄alkoxy;
and pharmaceutically acceptable salts thereof.

21. The method of claim 17, wherein said compound of formula (I), or a pharmaceutically acceptable salt thereof, is comprised in a pharmaceutical composition in association with a pharmaceutically acceptable carrier.
